(19) United States
(12) Patent Application Publication
(10) Pub. No.: US 2026/0118254 A1
(43) Pub. Date: Apr. 30, 2026

(54) ACOUSTIC FREQUENCY ATLAS-BASED APPROACH FOR PHOTOACOUSTIC IDENTIFICATION OF SURGICAL BIOMARKERS

(71) Applicant: THE JOHNS HOPKINS UNIVERSITY, Baltimore, MD (US)

(72) Inventors: Muyinatu BELL, Baltimore, MD (US); Eduardo GONZALEZ, Baltimore, MD (US)

(21) Appl. No.: 18/691,585

(22) PCT Filed: Sep. 16, 2022
(86) PCT No.: PCT/US2022/043903

§ 371 Date: Mar. 13, 2024

Related U.S. Application Data
(60) Provisional application No. 63/245,392, filed on Sep. 17, 2021.

(51) Int. Cl. G01N 21/17; G01N 33/49; G06N 3/09

(52) U.S. Cl. CPC G01N 21/1702; G01N 33/49; G06N 3/09

(57) ABSTRACT

A method for identifying biological markers using acoustic frequency response is disclosed. The method includes obtaining acoustic information from a received signal from a target location; classifying the acoustic information to provide classified acoustic information based on photoacoustic imaging response using a trained classifier; providing an indication of the classified acoustic information at the target location; and estimating concentration levels from mixtures of photoacoustic-sensitive materials.

### CROSS-REFERENCE TO RELATED APPLICATIONS

This application is the national stage entry of International Patent Application No. PCT/US2022/043903, filed on Sep. 16, 2022, and published as WO 2023/044075 A1 on Mar. 23, 2023, which claims the benefit of U.S. Provisional Patent Application Ser. No. 63/245,392, filed on Sep. 17, 2021, which are hereby incorporated by reference herein in their entireties.

### STATEMENT REGARDING FEDERALLY SPONSORED RESEARCH

This invention was made with Government support under grant no. EB018994, awarded by the National Institutes of Health, and under grant no. ECCS1751522, awarded by the National Science Foundation. The Government has certain rights in the invention.

### FIELD

This application is directed to photoacoustic identification of surgical biomarkers, and in particular, to systems and methods for acoustic frequency atlas-based approach for photoacoustic identification of surgical biomarkers.

### BACKGROUND

In photoacoustic imaging, spectral unmixing techniques are often employed to isolate signal origins (e.g., blood, contrast agents, lipids) with one goal of discriminating among critical chromophores during surgical interventions. These techniques comprise generating an overdetermined system of equations (i.e., more equations than variables) from the signal response of each chromophore at different laser wavelengths, which can then be solved with an optimization technique based on the known optical absorption coefficient for each chromophore at each wavelength. For example, Xia et al. used a pseudo inverse approach to differentiate photoacoustic responses originating from water, blood, and lipids, and Ding et al. investigated the effect of alternative versions with non-negativity constraints to determine the concentration levels of contrast agent injected in in vivo mice. More recently, Grasso et al. proposed an iterative approach to discriminate blood oxygenation levels by solving the system of equations with a nonnegative matrix factorization, which compensates for the ill-conditioned invertibility of the absorption coefficient matrix. Despite their effectiveness, these spectral unmixing techniques are typically not feasible for most real-time applications because of the lengthy acquisition times associated with transmitting multiple laser wavelengths to achieve a single estimate. Traditional spectral unmixing techniques also do not typically consider differences in acoustic spectra, which has the potential to provide additional information for differentiation between biomarkers or different soft tissues.

An alternative to optimization techniques is to consider an analysis of the acoustic spectra using spectral parameters. Initially, spectral parameters obtained from photoacoustic signals were used for characterization of tissues. For example, Kumon et al. conducted an in vivo study to detect prostate adenocarcinomas using the intercept, slope, and mid-band fit of the frequency response of photoacoustic RF signals, where the use of mid-band fit resulted in statistically significant differentiation between pathological and healthy tissue (p<0.01). Similarly, Strohm et al. used both the slope of a linear fit and the spectral peak to discriminate between concentrations of red blood cells. Later, Wang et al. used the slope parameter to accurately differentiate (p<0.01) the photoacoustic signals from particles of different diameters in phantom experiments. However, by reducing the dimensionality of the feature space, spectral parameter methods provide a limited snapshot of frequency characteristics. In addition, these methods use a calibration stage from a reference spectra whose source varies among studies (e.g., hair fibers, stainless steel blocks, gold-films, and black-dyed polymer micro-spheres), which limits the repeatability of classification performance for in vivo applications.

In contrast to spectral unmixing methods, two distinct approaches (i.e., F-mode imaging and a method proposed by Cao et al.) uses the complete acoustic spectra for differentiation of photoacoustic targets. F-mode imaging divides spectra with filter banks and displays a series of images of a specific frequency content, which are later combined with a label-free photoacoustic microscopy (PAM) map to selectively enhance the visualization of organelles. The method proposed by Cao et al. uses the acoustic spectra filtered with the frequency response of the ultrasound transducer to perform k-means clustering of photoacoustic signals originating from two different photoacoustic-sensitive materials. These two approaches share two limitations. First, in contrast to spectral unmixing techniques, labelled regions for each desired chromophore are required. Second, these labelled regions rely on a priori information about the location of materials to be differentiated. These limitations are not ideal for image guidance during surgical interventions and reduce overall classification performance.

### SUMMARY

According to examples of the present disclosure, a method for identifying biological markers using acoustic frequency response is disclosed. The method comprises obtaining acoustic information from a received signal from a target location; classifying the acoustic information to provide classified acoustic information based on photoacoustic imaging response using a trained classifier; and providing an indication of the classified acoustic information at the target location.

Various additional features can be included in the method including one or more of the following features. The method can further comprise providing a single wavelength radiation emission or a dual-wavelength emission to the target location. The trained classifier can be a neural network. The method can further comprise training a classifier to produce the trained classifier using a training data set of known photoacoustic-sensitive materials under a single wavelength emission conditions, a dual-wavelength emission conditions, or both. The training data set can comprise biological material and contrast agents. The biological material can comprise blood, lipid, collagen, or bone. The contrast agents can comprise methylene blue or indocyanine green (ICG). The method can comprise determining a concentration of each material within the target location based on the classified acoustic information. The method can further comprise alternative processing steps of the collected acoustic frequency information from two wavelength emission: difference of the spectra obtained from each wavelength emission; difference of the log compressed spectral obtained from each wavelength emission; and cross correlation between the spectra obtained from each wavelength emission. The method can further comprise of the use of a single light pulse containing two wavelengths for characterization of photoacoustic-sensitive materials through analysis of the frequency information of the acoustic response. In addition to single or two wavelength emission, the use of a single pulse comprising two wavelengths for excitation is expected to offer the following benefits: neglects the effect of motion artifacts in the imaging plane when switching between wavelength emissions, as only a single acquisition event is now configured; reduces the acquisition time of photoacoustic data which benefits the feasibility of the algorithm for real-time implementations; adds a third frequency response to the atlas which further enhances the differentiation between photoacoustic-sensitve materials; and adds robustness in the characterization of the frequency content by the comparison of the new frequency response to the responses obtained from individual wavelengths.

According to examples of the present disclosure, a system for identifying biological markers using acoustic frequency response is disclosed. The system comprises a hardware processor; a non-transitory computer readable medium that stores instructions that when executed by the hardware processor perform a method comprising: obtaining acoustic information from a received signal from a target location; classifying the acoustic information to provide classified acoustic information based on photoacoustic imaging response using a trained classifier; and providing an indication of the classified acoustic information at the target location.

Various additional features can be included in the system including one or more of the following features. The method further comprises providing a single wavelength radiation emission or a dual-wavelength emission to the target location. The trained classifier is a neural network. The method further comprises training a classifier to produce the trained classifier using a training data set of known photoacoustic-sensitive materials under a single wavelength emission conditions, a dual-wavelength emission conditions, or both. The training data set comprises biological material and contrast agents. The biological material comprises blood, lipid, collagen, or bone. The contrast agents comprise methylene blue and indocyanine green (ICG). The method further comprises determining a concentration of each material within the target location based on the classified acoustic information.

### BRIEF DESCRIPTION OF THE DRAWINGS

The accompanying drawings, which are incorporated in and constitute a part of this specification, illustrate embodiments of the present teachings and together with the description, serve to explain the principles of the present teachings. In the figures:

FIG. 1 shows an acquisition setup to test the differentiation of methylene blue (MB) from blood (Hb) according to examples of the present disclosure.

FIG. 2 shows an overview of a framework for differentiating photoacoustic signals sources according to examples of the present disclosure.

FIG. 3 shows two spectral analyses for characterization based on single (top) and dual (bottom) wavelength emissions according to examples of the present disclosure.

FIG. 4 shows a setup for reference spectra acquisition. A and B represent the imaging depths of 5 and 9 cm for aluminum, respectively. C and D represent the imaging depths of 5 and 9 cm for steel, respectively, according to examples of the present disclosure.

FIG. 5A, FIG. 5C, FIG. 5E, and FIG. 5G show locally-weighted short-lag spatial coherence (LW-SLSC) photoacoustic images overlaid on DAS ultrasound images of MB and Hb, obtained with a laser wavelength emission of (FIG. 5A) 730 nm (FIG. 5C), 800 nm (FIG. 5E) 890 nm, and (FIG. 5G) 920 nm according to examples of the present disclosure. FIG. 5B, FIG. 5D, FIG. 5F, and FIG. 5H show segmented masks for MB and Hb after a −6 dB threshold was applied to the LW-SLSC photoacoustic images according to examples of the present disclosure. (FIG. 5D) Compound mask from “OR” logical operation on masks generated from 690 nm to 800 nm. (FIG. 5H) Resulting mask from the “OR” logical operation of 690 nm and 920 nm masks.

FIG. 6 shows examples of segmented regions of MB and HB using different classification approaches according to examples of the present disclosure. The identified regions represent correctly classified pixels of MB, Hb, and also misclassified pixels. Each image shows the frame generated with the wavelength emission that achieved the highest accuracy.

FIG. 7 shows overall classification results with the dual-wavelength atlas method, single-wavelength atlas method, spectral unmixing methods, F-mode, and k-means clustering according to examples of the present disclosure. Top, middle and bottom row show the sensitivity, specificity and accuracy of classification, respectively. The left and right columns show the mean and standard deviation over 10 frames, respectively. For each image, the first 2 vertical stripes counting from the left represents the results for spectral unmixing and spectral unmixing+NNMF, respectively, which have a single value from 690 to 800 nm wavelengths. The next 3 stripes represent the results for the single wavelength atlas method, F-mode, and k-means clustering, respectively, as a function of wavelength emission. Finally, the upper triangle represents the results of the dual wavelength atlas method for each pair of wavelength combination.

FIG. 8 shows examples of stacked spectra of in-phase quadrature data from MB and Hb using (top) single-wavelength atlas method and (bottom) dual-wavelength atlas method according to examples of the present disclosure. The spectra show combined results obtained with 690 nm and 870 nm laser wavelength.

FIG. 9 shows a comparison of sensitivity and 1-specificity from single- and dual-wavelengths atlas method using a one frame per wavelength and wavelength pair, respectively, according to examples of the present disclosure. The threshold regions delimit cases with both sensitivity and specificity greater than 0.8, which represents a good classification performance.

FIG. 10 shows a classification accuracy of the spectral unmixing techniques for two testing datasets while varying the additive coefficient according to examples of the present disclosure.

FIG. 11 shows a reproducibility of classification accuracy between dual-wavelength atlas and spectral unmixing methods results, only specific hyper-parameters (see Eq. 7) would be selected and used in clinical practice according to examples of the present disclosure.

FIG. 12 shows the classification accuracy of k-means clustering when using different reference spectra presented as a stacked bar plot according to examples of the present disclosure.

FIG. 13A and FIG. 13B show the accuracy results of the spectral unmixing techniques when tested on Datasets 1-3 while varying the additive coefficient in Eq. 3 according to examples of the present disclosure.

FIG. 14A shows a summary of the accuracy results obtained with the spectral unmixing, single- and dual wavelength atlas, F-mode, and k-means clustering methods using the full range of wavelengths and hyperparameter γ, according to examples of the present disclosure.

FIG. 14B shows a subset of accuracy distributions obtained from the best 5 cases among the datasets, according to examples of the present disclosure.

FIG. 15 shows the classification accuracy of each dataset acquired with varying fluence pairs, according to examples of the present disclosure.

FIG. 16 shows the classification accuracy from the dual-wavelength atlas method tested on the datasets obtained with added tissue layers, according to examples of the present disclosure.

FIG. 17 shows criteria for determining the optimal number of principal components for the dual-wavelength atlas method using the (left) Kaiser rule (2) and (right) a 80% variance threshold, according to examples of the present disclosure.

FIG. 18 shows the mean accuracy of classification results for the dual-wavelength atlas method before (left) and after (right) parameter optimization, according to examples of the present disclosure

FIG. 19 shows a summary of sensitivity, specificity, and accuracy results of the dual-wavelength atlas method before and after parameter optimization, according to examples of the present disclosure.

FIG. 20 shows a mean classification accuracy of k-means clustering with different reference spectra, according to examples of the present disclosure.

FIG. 21A show an experimental setup and FIG. 21B shows a framework of the dual-wavelength atlas method for mixture estimation of methylene blue (MB) and Hb (Hb), according to examples of the present disclosure.

FIG. 22A, FIG. 22B, and FIG. 22C show the results from the photoacoustic spectra obtained with mixtures of MB and human Hb, according to examples of the present disclosure.

FIG. 23A and FIG. 23B show performance optimization of the dual-wavelength atlas algorithm using the human Hb dataset, according to examples of the present disclosure.

FIG. 24A and FIG. 24B show the estimated concentrations when using porcine Hb, according to examples of the present disclosure.

FIG. 25A and FIG. 25B shows examples of photoacoustic DAS images, coherence masks used for segmentation, and estimated concentration map for a ground-truth concentration of 60% (i.e., 60% MB and 40% experimental porcine Hb), according to examples of the present disclosure.

FIG. 26 shows examples of concentration maps obtained from one trial per ground-truth concentration results, according to examples of the present disclosure.

FIG. 27 shows a mean absolute error of the estimated chromophore concentration obtained with the dual-wavelength atlas method as a function of M-weighted SLSC coherence thresholds, according to examples of the present disclosure.

FIG. 28 shows examples of concentration maps generated with different coherence threshold values, according to examples of the present disclosure.

FIG. 29 illustrates a schematic view of a computing system that incorporates machine learning modules(s) to accomplish one or more tasks described in the present disclosure.

It should be noted that some details of the figures have been simplified and are drawn to facilitate understanding rather than to maintain strict structural accuracy, detail, and scale.

### DETAILED DESCRIPTION

Reference will now be made in detail to embodiments, examples of which are illustrated in the accompanying drawings and figures. In the following detailed description, numerous specific details are set forth in order to provide a thorough understanding of the invention. However, it will be apparent to one of ordinary skill in the art that the invention may be practiced without these specific details. In other instances, well-known methods, procedures, components, circuits and networks have not been described in detail so as not to unnecessarily obscure aspects of the embodiments.

To overcome challenges with traditional spectral unmixing, F-mode, and k-means clustering, a more general acoustic frequency-based analysis method is disclosed to discriminate photoacoustic responses from different materials. This method operates directly on the magnitude of the pressure signals to provide richer analysis information, presenting two key benefits. First, the method does not depend on a reference spectrum. Second, the method applies a classification framework using training and testing sets containing known photoacoustic-sensitive materials (i.e., no a priori signal location information is required). This method, which relies on an analysis of the acoustic frequency response from a single or dual-wavelength emission, is sufficient to differentiate biomarkers and has the potential to increase possible frame rates for real-time implementation in the operating room.

Spectral unmixing techniques for photoacoustic images are often used to isolate signal origins (e.g., blood, contrast agents, lipids). However, these techniques often require many (e.g., 12-59) wavelength transmissions for optimal performance to exploit the optical properties of different biological chromophores. Analysis of the acoustic frequency response of photoacoustic signals has the potential to provide additional discrimination of photoacoustic signals from different materials, with the added benefit of potentially requiring only a few optical wavelength emissions. This disclosure presents results testing this hypothesis in a phantom experiment, given the task of differentiating photoacoustic signals from deoxygenated hemoglobin (Hb) and methylene blue (MB). Coherence-based beamforming, principal component analysis, and nearest neighbor classification were employed to determine ground-truth labels, perform feature extraction, and classify image contents, respectively. The mean±one standard deviation of classification accuracy was increased from 0.63±0.13 to 0.82±0.10 when increasing the number of wavelength emissions from one to two, respectively. When using an optimal laser wavelength pair of 690 nm and 870 nm, the sensitivity and specificity of detecting MB over Hb were 1.00 and 1.00, respectively. Results are highly promising for the differentiation of photoacoustic-sensitive materials with comparable performance to that achieved with more conventional multispectral laser wavelength approaches.

To test our hypothesis, a frequency analysis was applied to the received photoacoustic signals from two chromophores-blood and methylene blue. The necessity to differentiate these two chromophores is motivated by photoacoustic-guided hysterectomy techniques that require differentiation of uterine arteries from ureters containing methylene blue (10). Although the focus of this disclosure is the distinction of these two chromophores, the disclosed photoacoustic differentiation is applicable to other chromophores of interest during a surgical procedure.

The remainder of this section is organized as follows. Section 2 details acquisition, segmentation, and classification methods to identify photoacoustic signals originating from either methylene blue or blood, followed by summaries of existing methods used to benchmark the performance of our approach on the same datasets. Section 3 presents the quantitative and qualitative comparison of the classification performance between the disclosed and the existing methods. Section 4 discusses insights from these results, and Section 5 summarizes our conclusions.

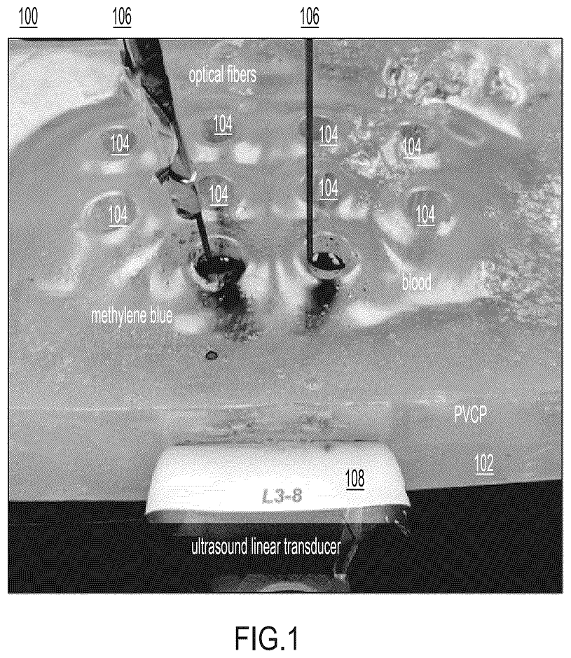

FIG. 1 shows an acquisition setup 100 to test the differentiation of methylene blue (MB) from blood (Hb) according to examples of the present disclosure. These photoacoustic-sensitive materials fill the hollow chambers of a custom polyvinyl chloride plastisol phantom.

### 2. METHOD

### 2.1. Acquisition Setup

As shown in FIG. 1, a polyvinyl chlorideplastisol (PVCP) phantom 102, such as a 29 cm×18 cm×10 cm (length×width×height) PVCP phantom, comprises a plurality of cylindrical hollow chambers 104, such as ten cylindrical, hollow chambers. For example, each chamber has a diameter of 15 mm and a depth of 55 mm. Two of the hollow chambers were filled with a 1% weight-by-volume aqueous solution of methylene blue (MB) and human blood (Hb), and an optical fiber 106, such as a 1-mm-diameter optical fiber, was inserted in each of the filled chambers. These fibers originated from a bifurcated fiber bundle that was connected to a Phocus Mobile laser (Opotek Inc., Carlsbad, CA, USA), transmitting laser light with wavelengths ranging from 690 nm to 950 nm in 10 nm increments. The tip of each optical fiber was positioned approximately 15 mm below the top surface of the chambers, and photoacoustic signals were generated with an energy of 4 mJ at each fiber tip. By transmitting light locally into each chamber and not globally illuminating multiple chambers simultaneously, the differences in fluence and the related amplitude of responses to the optical excitation are minimized. The generated photoacoustic signals were received by an Alpinion L3-8 linear array ultrasound probe 108 (center frequency of 5.5 MHz and pitch of 0.3 mm) that was positioned on the lateral wall of the phantom, approximately 40 mm away from the hollow chamber cross section, as shown in FIG. 1.

To evaluate the reproducibility of the disclosed method, two datasets were acquired. Dataset 1 comprises training and testing sets that were acquired 4 days apart to mimic an approach that requires training several days before surgery. The samples were stored with interim refrigeration at a temperature of 4° C. Dataset 2 comprises training and testing sets acquired on the same day to ensure that time lapse was not an important variable affecting results obtained with Dataset 1. Dataset 2 was used for the majority of assessments.

### 2.2. Ground-Truth Labelling

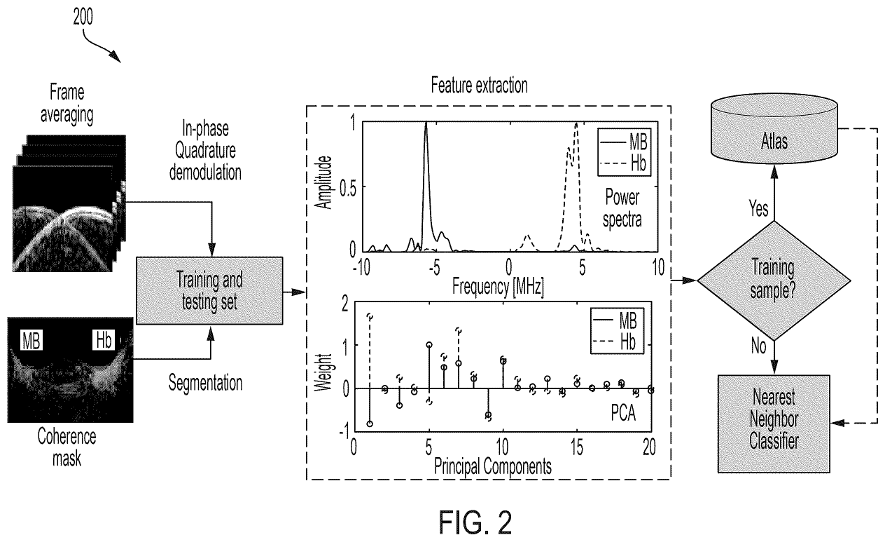

FIG. 2 shows an overview of a system for differentiating photoacoustic signals sources 200 according to examples of the present disclosure. For each laser wavelength emission, 10 acquisitions of raw radiofrequency data were acquired. Photoacoustic images were then generated using conventional delay-and-sum (DAS) beamforming. Two regions of interest (ROIs) were defined to separate photoacoustic signals generated from MB and Hb, located on the right and the left sides of the photoacoustic images, respectively. Ground-truth labels were segmented from locally weighted short-lag spatial coherence (LW-SLSC) images (11), using a regularization factor of α=1 and an axial correlation kernel of 2λ, where λ is the wavelength associated with the center frequency of the L3-8 ultrasound probe. Binary segmentation was performed using a −6 dB threshold mask applied to the LW-SLSC images. A single binary mask was computed per laser wavelength emission, which was the result of the logical inclusive “OR” operation of the 10 masks obtained from the 10 frames. For each image, only those pixels included in the coherence masks were used for feature extraction, training, and classification.

### 2.3. Atlas of Photoacoustic Sensitive Materials

A frequency analysis of the magnitude of the photoacoustic pressure waves was performed. This analysis required computation of the absolute value of the raw signals (rather than the signals conventionally used for beamforming, which have negative and positive values), as the shape of the spectra provides more distinctive features between photoacoustic-sensitive materials.

For each material (i.e., MB and Hb), the normalized power spectra were calculated from a sliding window of axial kernels of in-phase and quadrature (IQ) data (which were converted to baseband with a modulation frequency of 2.5 MHz and a band-pass filter of 200% bandwidth), each 3.85 mm in length. Principal component analysis (PCA) was applied to the power spectra of photoacoustic signals acquired at each laser wavelength in order to reduce the length of each power spectra to its first 20 principal components. When using a training set, these principal components were stored in an “atlas” describing each material. Finally, when evaluating the spectra of an unknown signal, nearest neighbor (NN) classification was applied with the L2-norm as the measure of distance between the PCA of the unknown spectra and the atlas.

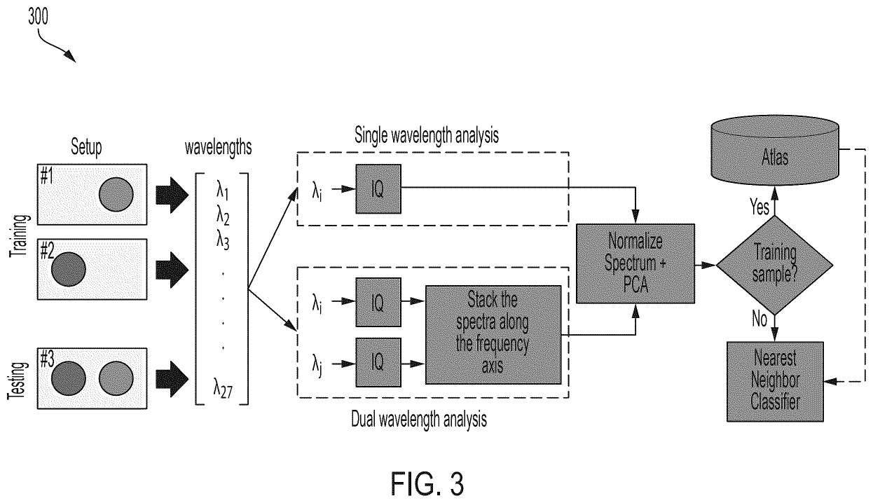

FIG. 3 shows two spectral analyses for characterization based on single (top) and dual (bottom) wavelength emissions 300 according to examples of the present disclosure. In the dual-wavelength analysis, the IQ spectra of the photoacoustic response from a region of interest using two different wavelengths were stacked and normalized to enhance differences in frequency content. No stacking was required for the single-wavelength analysis.

2.4. Comparison with Previous Methods

### 2.4.1. Spectral Unmixing Techniques

Spectral unmixing techniques solve the source component reconstruction C of a scanned region by using the multispectral measurement matrix M and an a priori absorption coefficient matrix S of the number of chromophore present in the image. A conventional spectral unmixing solution is the least square method presented below (1; 2; 12):

$$C = {MS}^{†} , C ∈ {ℝ}^{n \times k} , M ∈ {ℝ}^{n \times m} , {S}^{†} ∈ {ℝ}^{m \times k} , \qquad ( 1 )$$

where C segments k number of chromophores over a grid of n pixels, m is the number of laser wavelength acquisitions, and S is the Moore-Penrose pseudoinverse of the absorption coefficient matrix S (i.e., S^{†}+=S^{T} (SS^{T})⁻¹). A more robust approach proposed by Grasso et al. (4) used an iterative non-negative matrix factorization (NNMF) to adjust the initial S and further reduce residual errors:

$$[ C , \hat{S} ] = {min}_{C , S} \frac{1}{2} {‖ M - CS ‖}_{2}^{2} , S ∈ {ℝ}^{k \times m} \qquad ( 2 )$$

$${C}_{ij} \geq 0 , {S}_{ij} \geq 0$$

$$S ( p + 1 ) = S ( p ) ⊗ \frac{( {W}^{T} M )}{( {C}^{T} CS )}$$

$$C ( p + 1 ) = C ( p ) ⊗ \frac{( {MS}^{T} )}{( {CSS}^{T} )}$$

where ⊗ and the division are considered element-wise operations and the stopping criteria is defined by an improvement tolerance n. Eq. 1 and Eq. 2 are considered overdetermined systems (i.e., m>k). Both methods were applied to DAS images of the testing data described in Section 2.2 and FIG. 3 for two chromophores (i.e., k=2). However, because there is no report of absorption coefficients for methylene blue at optical wavelengths greater than 800 nm, only 12 of the 27 wavelength acquisitions were used for the construction of the matrix M (i.e., m=12). In addition, each equation was regularized by modifying the initial S to:

$${S}^{′} = S + γ A , \qquad ( 3 )$$

where A is a matrix of ones, and γ is a positive constant that was varied from 10⁻¹ to 10³ cm⁻¹ in 50 exponential increments.

TABLE 1
Wavelengths and hyper-parameters evaluated

| | Wavelengths | Hyper- |
|---|---|---|
| Method | evaluated | Parameter |
| Spectral unmixing | 690-800 nm | Γ |
| Spectral unmixing + | 690-800 nm | Γ |
| NNMF | | |
| F-mode imaging | 690-950 nm | — |
| k-means clustering | 690-950 nm | reference |
| | | spectra |
| Single-wave atlas | 690-950 nm | — |
| — | — | — |

### 2.4.2. F-Mode Imaging

Using the testing data, non-normalized DAS images were generated without log-compression and segmented with the coherence mask described in Section 2.2. Log-compressed power spectra were calculated from a sliding window of axial kernels of radiofrequency signals, each 3.85 mm in length. For each spectrum, the integrated frequency content was estimated from 4 sectors of the frequency domain of 0.2 MHz width and center frequencies of 1, 2, 3, and 4 MHz. Then, for each segmented DAS image, k-means clustering was applied to separate MB and Hb axial kernels. This process was repeated for each single wavelength acquisition and each frame.

2.4.3. Acoustic-Based Clustering with Calibrated Spectra

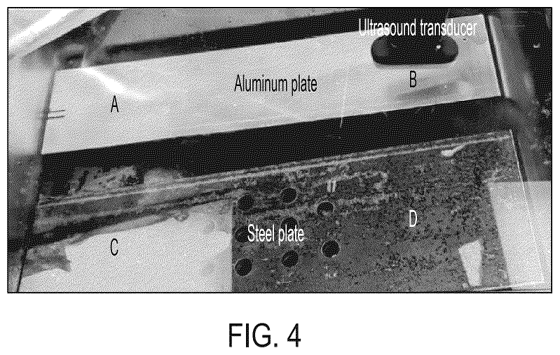

To perform acoustic-based clustering, the spectra of each radio-frequency axial kernel were first calibrated to a reference spectrum that models the characteristic frequency response of the ultrasound system. To evaluate performance variability, eight different ultrasound reference spectra were acquired from two materials: (1) an aluminum plate and (2) a carbon-steel plate. The ultrasound acquisition setup for these two flat, reflective surfaces, which make them ideal for use as reference spectra (13), are shown in FIG. 4. These two materials have acoustic properties that produce different pulse-echo responses (14; 15). The acquisitions were obtained after independently placing each material at axial depths of 5 cm or 9 cm, and tilted 45 degrees or 0 degrees relative to the elevation-lateral imaging plane. Each reference spectra was calculated by averaging the fast Fourier transform of scan lines across the lateral dimension. The generation of the log spectra was similar to that of F-mode imaging. However, each spectrum was then calibrated over a single reference spectrum and then further normalized at 0 dB. Finally, k-means clustering was conducted for classification, and the process was repeated for each single-wavelength acquisition and each frame.

FIG. 4 shows a setup for reference spectra acquisition. A and B represent the imaging depths of 5 and 9 cm for aluminum, respectively. C and D represent the imaging depths of 5 and 9 cm for steel, respectively, according to examples of the present disclosure.

2.5. Evaluation of laser wavelength and hyper-parameters Table 1 summarizes the range of light-emission wavelengths used for each method as well as the corresponding hyperparameter to further improve the robustness of the classification performance.

The regularization coefficient represents a tradeoff between classification performance and reproducibility for the spectral unmixing methods, as the condition number of matrix S′ increases when increases, and the system becomes more ill-posed. The variation of the reference spectra evaluates the consistency of the classifications results for the k-means clustering method when considering different materials and acquisition setups.

2.6. Classification performance metrics MB and Hb were considered to be the positive and negative samples, respectively, when calculating sensitivity, specificity, and accuracy metrics of classification performance. Sensitivity or true positive rate (TPR) measures the fraction of pixels that were correctly classified as methylene blue:

$$Senstivity = TPR = \frac{{T}_{MB}}{{T}_{MB} + {F}_{Hb}} \qquad ( 4 )$$

where T_{MB} and F_{Hb} are the number of true MB and false Hb pixels, respectively. Similarly, specificity or true negative rate (TNR) measures the fraction of pixels that are correctly classified as deoxygenated blood:

$$Specificity = TNR = \frac{{T}_{Hb}}{{T}_{Hb} + {F}_{MB}} , \qquad ( 5 )$$

where T_{Hb} and F_{MB} are the number of true Hb and false MB pixels, respectively. The combination of sensitivity and specificity is described by the accuracy metric, defined as:

$$Accuracy = ACC = \frac{{T}_{MB} + {T}_{Hb}}{{T}_{MB} + {T}_{Hb} + {F}_{MB} + {F}_{Hb}} , \qquad ( 6 )$$

To determine the optimal parameter for each method, the three quantitative metrics were considered simultaneously, using the optimization expression:

$$λ = {max}_{λ} f ( λ ) , f ( λ ) = \frac{1}{2} ( \frac{TPR ( λ ) + TNR ( λ )}{2} + ACC ( λ ) ) , \qquad ( 7 )$$

where λ is either the optimal single-wavelength for the single wavelength atlas method, k-means clustering or F-mode imaging method, the optimal pair of wavelengths for the dual-wavelength atlas method, or the optimal γ constant for the spectral unmixing methods.

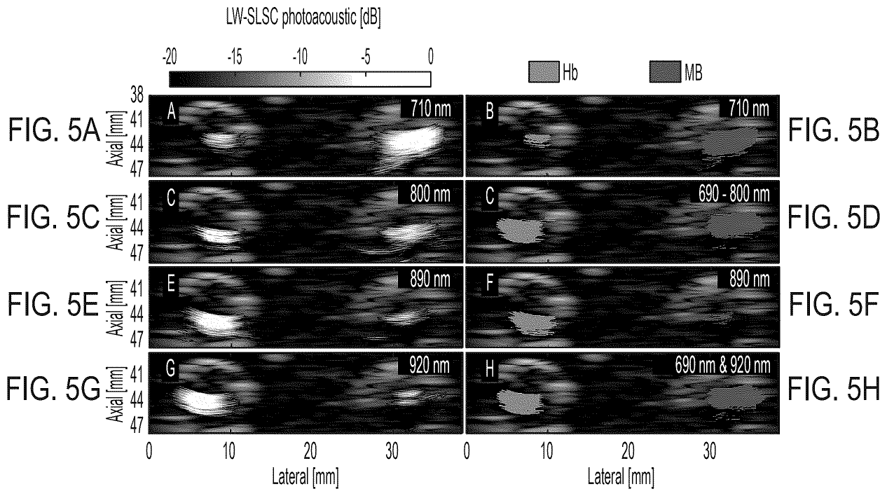

FIG. 5A, FIG. 5C, FIG. 5E, and FIG. 5G show locally-weighted short-lag spatial coherence (LW-SLSC) photoacoustic images overlaid on DAS ultrasound images of MB and Hb, obtained with a laser wavelength emission of (FIG. 5A) 730 nm (FIG. 5C), 800 nm (FIG. 5E) 890 nm, and (FIG. 5G) 920 nm according to examples of the present disclosure. FIG. 5B, FIG. 5D, FIG. 5F, and FIG. 5H show segmented masks for MB and Hb after a −6 dB threshold was applied to the LW-SLSC photoacoustic images according to examples of the present disclosure. (FIG. 5D) Compound mask from “OR” logical operation on masks generated from 690 nm to 800 nm. (FIG. 5H) Resulting mask from the “OR” logical operation of 690 nm and 920 nm masks.

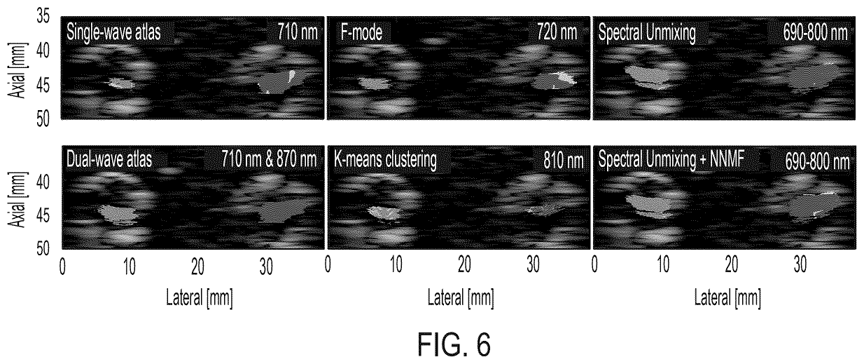

FIG. 6 shows example of segmented regions of MB and HB using different classification approaches according to examples of the present disclosure. The identified regions represent correctly classified pixels of MB, Hb, and also misclassified pixels. Each image shows the frame generated with the wavelength emission that achieved the highest accuracy.

Finally, a pair-wise t-test was used to evaluate the statistical significance (p<0.01) of the difference between MB and Hb spectra obtained from either the single-wavelength atlas or dual-wavelength atlas method.

### 3. RESULTS

### 3.1. Image and Segmentation Examples

The left column of FIG. 5 shows example LW-SLSC photoacoustic images co-registered to a DAS ultrasound image obtained with the laser wavelength indicated on each image. The right column of FIG. 5 shows the corresponding segmentation mask, where the blue and red regions represent the ground truth labeled pixels for MB and Hb, respectively. FIG. 5(d) shows an example compound masks generated by the “OR” logical operation of a range of masks obtained from 690 nm to 800 nm wavelength. FIG. 5(h) shows an example compound masks generated by the “OR” logical operation from a mask pair obtained with 690 nm and 620 nm wavelengths, respectively. Note that the varying area of the LW-SLSC signals and corresponding masks for MB and Hb regions for different laser wavelength emissions is responsible for different proportions of MB-to-Hb kernel sizes when calculating the quantitative metrics.

FIG. 6 shows segmentation examples of the best results for each of the classification approaches after estimating the corresponding optimal parameter, using the optimization expression defined by Eq. (7). The identified regions represent correctly classified pixels of MB and Hb, and also misclassified pixels. As observed previously in FIG. 5, the changes in region size among the approaches are caused by the different LW-SLSC coherence masks that were computed for either single, pairs, or groups of wavelengths, depending on the requirements of each classification method. For the k-means clustering method, the reference spectra from the flat steel plate at 9 cm was chosen, as it reported the highest overall accuracy among the emitted wavelengths (0.54±0.03). When qualitatively comparing the classified regions, the dual-wavelength atlas method showed the best classification performance, as the majority of each MB and Hb region were labelled correctly (i.e., no regions are shown). Similar performance was observed for the F-mode imaging method, the spectral unmixing, and spectral unmixing+NNMF, where the last two were generated with a coefficient of 828.6 and 7.54 cm⁻¹, respectively. Conversely, both spectral k-means clustering and single-wavelength atlas method failed to adequately detect signals from Hb, with a specificity of 0.56 and 0.59, respectively. A summary of the sensitivity, specificity and accuracy obtained with the optimal parameter for each method is shown in Table 2.

TABLE 2
Best classification case

| Method | Sensitivity | Specificity | Accuracy |
|---|---|---|---|
| Spectral unmixing | 0.95 | 0.80 | 0.88 |
| Spectral unmixing + | 0.97 | 0.85 | 0.91 |
| NNMF | | | |
| F-mode imaging | 0.83 | 1.00 | 0.88 |
| k-means clustering | 0.84 | 0.56 | 0.64 |
| Single-wave atlas | 0.96 | 0.59 | 0.86 |
| Dual-wave atlas | 1.00 | 1.00 | 1.00 |

### 3.2. Comparison of Sensitivity, Specificity, and Accuracy

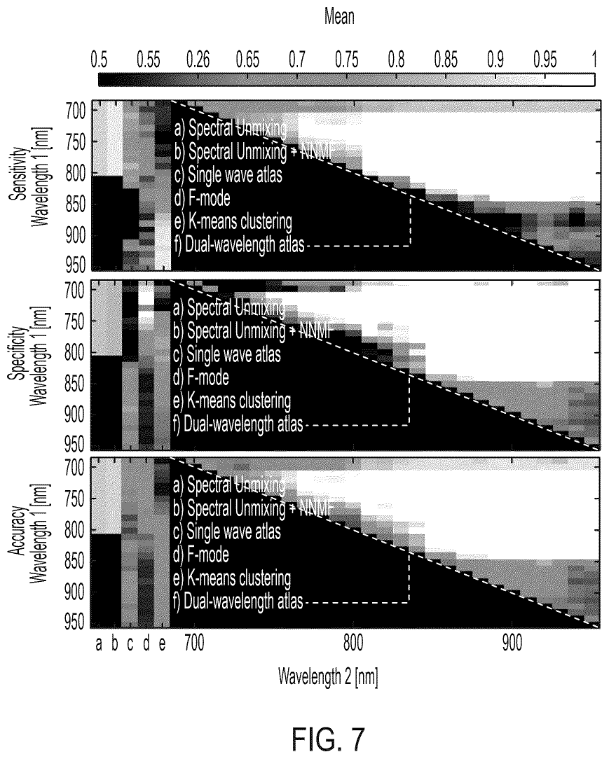

FIG. 7 shows overall classification results with dual-wavelength atlas, single-wavelength atlas, spectral unmixing methods, F-mode, and k-means clustering according to examples of the present disclosure. Top, middle and bottom row show the sensitivity, specificity and accuracy of classification, respectively. The left and right columns show the mean and standard deviation over 10 frames, respectively. For each image, the first 2 vertical stripes counting from the left represents the results for spectral unmixing and spectral unmixing+NNMF, respectively, which have a single value from 690 to 800 nm wavelengths. The next 3 stripes represent the results for single wave atlas, F-mode, and k-means clustering, respectively, as a function of wavelength emission. Finally, the upper triangle represents the results of the dual wave atlas for each pair of wavelength combination.

Both spectral unmixing techniques were computed with a γ=7:54 cm⁻¹, where spectral unmixing and spectral unmixing+NNMF achieved mean accuracies of 0.561 and 0.904, respectively.

Defining a minimum accuracy of 0.80 as good classification, the single-wavelength atlas method showed good classification performance at 750 nm wavelength while F-mode imaging achieved good classification performance at wavelengths lower than 830 nm. A poor overall classification performance was observed for k-means clustering, where the accuracy values were less than 0.6 from wavelengths of 690 nm to 950 nm. In contrast, the majority of wavelength pair combinations met the criteria for the dual-wavelength atlas method, with the exception of pairs generated when both wavelengths were less than 790 nm.

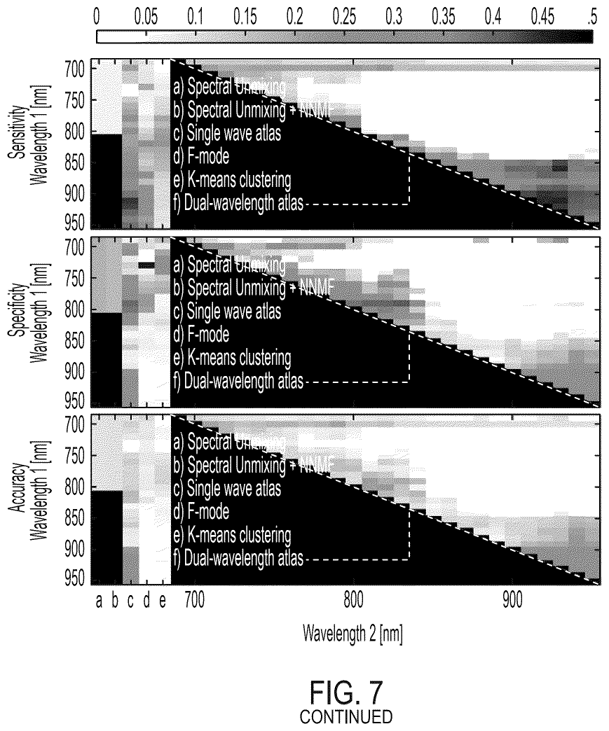

FIG. 8 shows examples of stacked spectra of in-phase quadrature data from MB and Hb using (top) single-wavelength atlas method and (bottom) dual-wavelength atlas method according to examples of the present disclosure. The spectra show combined results obtained with 690 nm and 870 nm laser wavelength.

As it is equally important to identify both MB and Hb regions, a high sensitivity and a low specificity pair, or vice-versa, corresponds in practice to a poor classification performance. Therefore, the totality of graphs shown in FIG. 7 is analyzed from this perspective. For some accuracy regions that are shown (i.e., adequate classification), the same regions are identified (i.e., poor classification) for either the corresponding specificity or sensitivity (i.e., good classification) for the remaining metric. For example, F-mode imaging presented accuracy values greater than 0.75 in the wavelength range of 810-840 nm, while presenting specificity values less than 0.40 over the same wavelength range. Similarly, the k-means clustering method showed a mean±one standard deviation sensitivity of 0.86±0.12 over the wavelength range of 710-800 nm, while showing a specificity and accuracy of 0.41±0.05 and 0.55±0.03, respectively, over the same wavelength range. These results further support the importance of evaluating sensitivity, specificity, and accuracy simultaneously, as described by Eq. 7, to determine the overall classification performance of each method. The mean±one standard deviation for this combination of accuracy, sensitivity, and specificity (i.e., f in Eq. 7), over the same wavelength ranges described above for the F-mode and the k-means clustering methods were 0.68±0.10 and 0.59±0.03, respectively.

TABLE 3
Average among wavelengths

| Method | Sensitivity | Specificity | Accuracy |
|---|---|---|---|
| Spectral unmixing | 0.95 ± 0.07 | 0.43 ± 0.33 | 0.67 ± 0.15 |
| Spectral unmixing + | 0.81 ± 0.19 | 0.42 ± 0.25 | 0.60 ± 0.12 |
| NNMF | | | |
| F-mode imaging | 0.76 ± 0.16 | 0.78 ± 0.20 | 0.71 ± 0.07 |
| k-means clustering | 0.64 ± 0.20 | 0.48 ± 0.18 | 0.54 ± 0.03 |
| Single-wave atlas | 0.78 ± 0.12 | 0.49 ± 0.13 | 0.63 ± 0.13 |
| Dual-wave atlas | 0.92 ± 0.17 | 0.71 ± 0.16 | 0.82 ± 0.10 |

Average among the range of γ values reported in Section 2.4.1

TABLE 4
Average among best 5 cases

| Method | Sensitivity | Specificity | Accuracy |
|---|---|---|---|
| Spectral unmixing | 0.96 ± 0.00 | 0.80 ± 0.01 | 0.88 ± 0.00 |
| Spectral unmixing + | 0.97 ± 0.00 | 0.84 ± 0.00 | 0.91 ± 0.00 |
| NNMF | | | |
| F-mode imaging | 0.74 ± 0.05 | 0.99 ± 0.00 | 0.83 ± 0.03 |
| k-means clustering | 0.67 ± 0.22 | 0.69 ± 0.21 | 0.61 ± 0.02 |
| Single-wave atlas | 0.90 ± 0.06 | 0.65 ± 0.10 | 0.82 ± 0.02 |
| Dual-wave atlas | 1.00 ± 0.00 | 1.00 ± 0.00 | 1.00 ± 0.00 |

only one best case per wavelength, wavelength pair, or γ

Tables 3 and 4 present summaries of quantitative metrics from the average among wavelengths and the average among the best 5 cases of each classification method, respectively. The quantitative results follow the same trend as those described in the qualitative results, where the dual-wave atlas method achieved the highest sensitivity, specificity, and accuracy among the other methods. Similarly, the method with the second highest average in the best-5-cases evaluation was the spectral unmixing+NNMF.

### 3.3. Spectra Examples

FIG. 8 shows a representative example of spectra from the dual-wavelength atlas method and the equivalent stacked spectra from the single-wavelength atlas method, in efforts to understand the improvement in classification performance when using the dual-wavelength atlas method. For this example, the testing spectra of 690 nm and 870 nm wavelengths were used, as this wavelength pair was one of five to achieve a specificity and sensitivity of 1.00 when using the dual-wavelength atlas method. The mean and standard deviation of the spectra were calculated from the spectra of all segmented pixels of MB and Hb data acquired with 690 nm or 870 nm wavelength for each atlas method. The overlapping spectra with the single wavelength atlas method applied to either 690 nm or 870 nm wavelength acquisitions (top of FIG. 8) resulted in no statistically significant differences between the amplitude of the spectra for MB and Hb (p>0.001), while Hb and MB differentiation was achieved with statistical significance (p<0.001) when using the dual-wavelength atlas method (bottom of FIG. 8). This example illustrates that the enhanced differentiation achieved with the dual-wavelength atlas method can be attributed to the ability to differentiate the two spectra.

### 3.4. Multiple Possible Dual-Wavelength Combinations

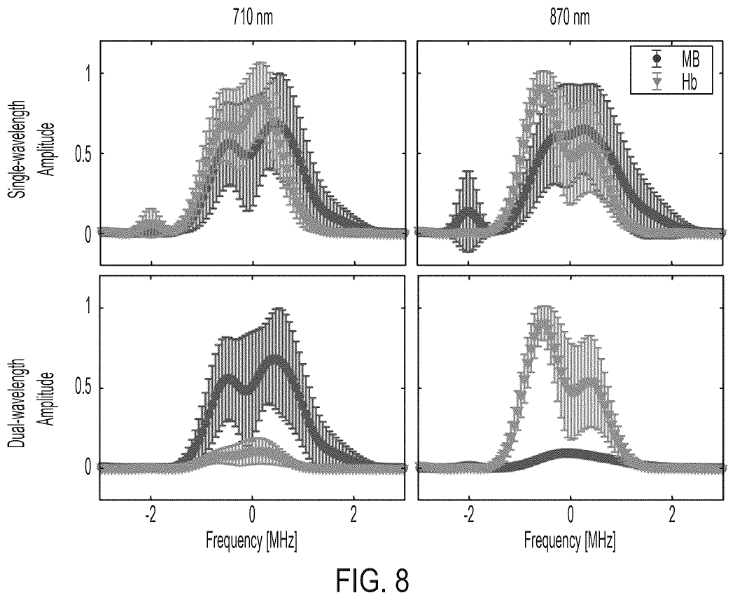

The results shown in FIG. 7 and Table 4 suggest that the dual-wavelength atlas method achieves high classification performance from a range of wavelengths combinations, whereas the other methods achieve their highest classification performance from just a few cases. To further visualize this claim, FIG. 9 shows a comparative evaluation of sensitivity and specificity between the atlas methods in an ROC-curve format. For example, when a threshold region of 0.80 sensitivity and 0.80 specificity is defined as the criterion for adequate classification, 85 and 0 wavelengths pairs were found for the dual- and single wavelength atlas methods, respectively. Therefore, the dual wavelength atlas method provides a flexible range of light emission wavelength pairs such that the ideal pair can be chosen to differentiate between the same chromophores across multiple imaging environments. This flexibility is necessary when an unwanted chromophore produces a considerable photoacoustic response at the originally selected wavelength pair.

FIG. 9 shows a comparison of sensitivity and 1-specificity from single- and dual-wavelengths atlas method using a one frame per wavelength and wavelength pair, respectively, according to examples of the present disclosure. The threshold regions delimits cases with both sensitivity and specificity greater than 0.8, which represents a good classification performance.

### 3.5. Dataset Sensitivity

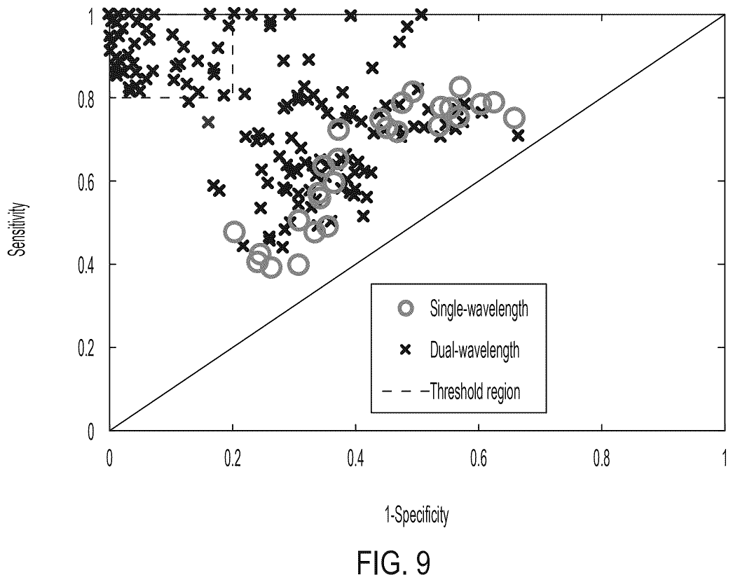

FIG. 10 shows a classification accuracy of the spectral unmixing techniques for two testing datasets while varying the additive coefficient according to examples of the present disclosure.

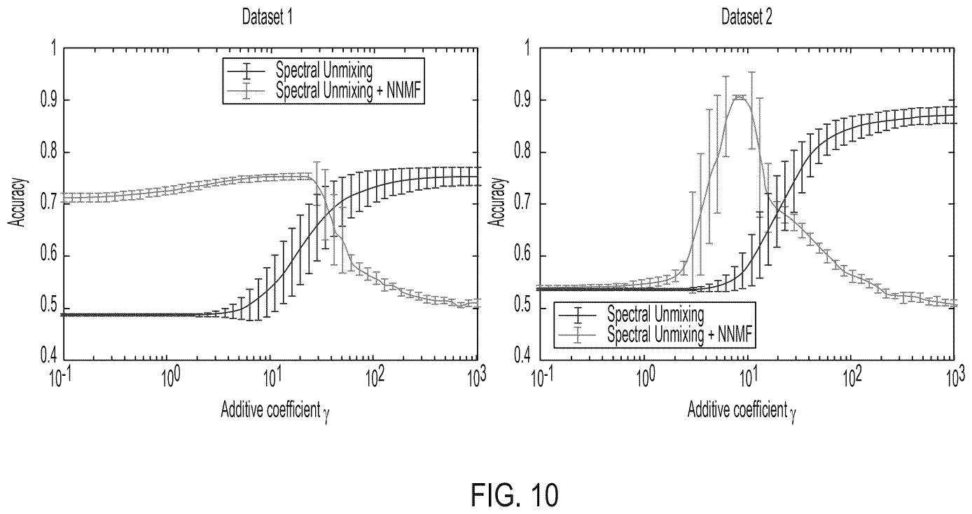

FIG. 11 shows a reproducibility of classification accuracy between dual-wavelength atlas and spectral unmixing methods results, only specific hyper-parameters (see Eq. 7) would be selected and used in clinical practice according to examples of the present disclosure.

While the spectral unmixing+NNMF method reported an improvement in the classification performance over the conventional spectral unmixing method, as shown in Table 2 and 4, the calculation of the optimal hyperparameter for the NNMF method was sensitive to the dataset used. FIG. 10 shows the accuracy results of the spectral unmixing techniques for the two testing datasets while varying the additive coefficient in Eq 3. Overall, spectral unmixing+NNMF showed the highest accuracy for Datasets 1 and 2 with a mean±one standard deviation of 0.75±0.01 and 0.90±0.01, respectively. However, the optimal parameter that generated the highest accuracy in spectral unmixing+NNMF was different among datasets, which were 23.3 cm⁻¹ and 7.54 cm⁻¹ for Dataset 1 and 2, respectively. Conversely, a high γ guaranteed the best classification performance for the conventional spectral unmixing method as the accuracy converged to a steady state value in both datasets. By fixing γ to 7.54⁻¹ (i.e., the optimal parameter chosen for FIG. 7), spectral unmixing+NNMF showed a mean change in accuracy of 0.155, while the conventional spectral unmixing showed a change in accuracy of 0.041. Two observations follow from these results. First, a high classification performance was not achieved with spectral unmixing+NNMF for multiple datasets without changing the regularization coefficient. Second, the use of different datasets influences the estimation of the optimal parameter and changes the maximum performance achieved with the spectral unmixing methods.

FIG. 11 shows a summary of the accuracy results obtained with the dual-wavelength atlas, spectral unmixing, and spectral unmixing+NNMF methods. For each dataset, the box plots show the accuracy from 351 wavelength pairs for dual-wavelength atlas method, the highest 500 accuracy values for the spectral unmixing method, and the highest 130 accuracy values for the spectral unmixing+NNMF method. When changing from Dataset 1 to Dataset 2, the median value decreased by 0.069 for dual-wavelength atlas method, increased by 0.107 for spectral unmixing, and decreased by 0.059 for spectral unmixing+NNMF. While the dual-wavelength atlas and spectral unmixing methods show a wide range of accuracy

As shown in FIG. 11 for the dual-wavelength atlas method, the maximum accuracy remained unchanged with an accuracy of 1.00, while this metric differed between the two datasets by 0.091 and 0.149 for the spectral unmixing and spectral unmixing+NNMF methods, respectively. In addition, for the dual-wavelength atlas method in both datasets, the 690 and 870 nm wavelength pair was present among the 5 pairs of wavelengths with the highest accuracy, both with a sensitivity, specificity, and accuracy of 1.00, respectively. Therefore, the dual-wavelength atlas method shows higher reproducibility of classification performance than spectral unmixing methods.

### 3.6. Reference Spectra Sensitivity

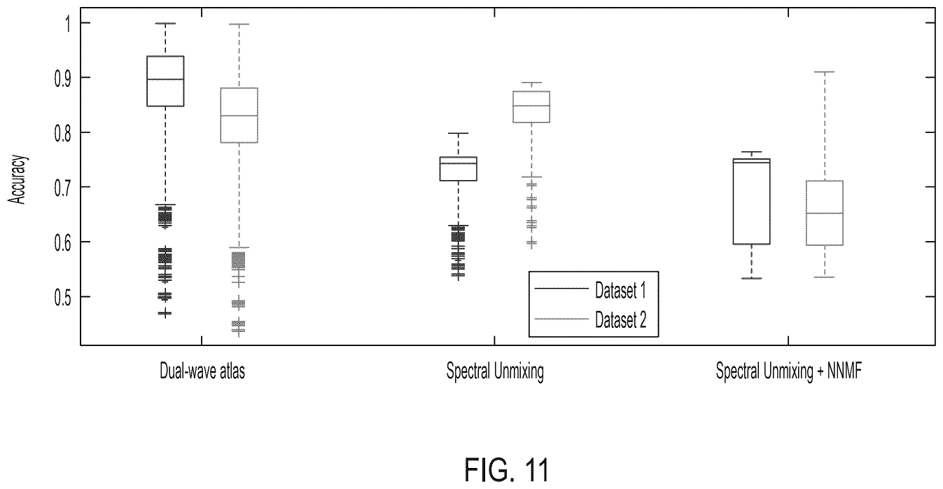

FIG. 12 shows the classification accuracy of k-means clustering when using different reference spectra described in Section 2.4.3, presented as a stacked bar plot. The optimal parameter, defined by Eq. 7, for the k-means clustering method used in FIG. 7 was the reference spectra obtained from the carbon-steel plate tilted 0° along the lateral imaging depth and located at an imaging depth of 9 cm. When computing the mean±one standard deviation of the difference in accuracy between a pair of results from different reference spectra, the lowest value was found between the tilted aluminum plate reference at 9 cm depth and flat steel plate reference at 5 cm depth with 0.0019±0.0017. Similarly, the highest difference was found between the flat aluminum plate reference at 9 cm depth and flat steel plate reference at 9 cm depth with 0.0099±0.0081. This last difference showed a maximum variation of 5.45% in classification performance at 750 nm when switching between reference sources. Despite the low sensitivity, specificity, and accuracy reported in Table 2, 3, 4, and, a difference of 5.45% in classification performance can be further increased by using more sophisticated materials as described in (5-7).

FIG. 12 shows mean classification accuracy of k-means clustering with different reference spectra, represented as a stacked bar plot to compactly display this information and to avoid overlap of mostly similar accuracy values at each wavelength.

### 4. DISCUSSION

A method to accurately identify biological markers by analyzing the acoustic frequency response from either a single-wavelength emission (i.e., single wavelength atlas method) or two consecutive wavelength emissions (i.e., dual-wavelength atlas method) has been provided. Overall, the best classification accuracy obtained with the dual-wavelength atlas approach outperforms that obtained from previous methods with similar goals, including spectral unmixing, F-mode imaging, and k-means clustering.

Normalization plays a key role in the single-wavelength atlas, dual-wavelength atlas, and k-means clustering methods because it prevents the use of amplitude as a distinguishing feature for classification. This is particularly important when characterizing structures located at different distances from the light source. By normalizing the spectrum from a single wave, both single-wavelength atlas and k-means clustering algorithms rely purely on the frequency shape for tissue differentiation, which is often challenged by the limited frequency content obtained with a limited-bandwidth ultrasound transducer. In contrast, the dual-wavelength atlas method normalizes a pair of spectra, removing the amplitude dependency between two different regions but at the same time preserving the relative amplitude difference of two different light emission responses from the same region.

In clinical practice, dual excitation wavelengths illuminating the region of interest with a fast-switching laser source that quickly alternates between wavelengths can be used, providing real-time labeling of photoacoustic-sensitive regions with comparable performance to that achieved with more conventional spectral unmixing techniques. The disclosed method can be beneficial for a range of emerging photoacoustic imaging approaches oriented to surgeries and interventions, such as hysterectomies, neurosurgeries, spinal fusion surgeries, as well as identification and distinction of metallic tool tips, cardiac catheter tips, and needle tips from other surrounding structures of interest.

The currently sequential comparison of measurements with atlas spectra can potentially be addressed with at least one of three possible strategies. First, graphical processing units (GPUs) may be employed for concurrent comparisons of a test spectrum with several training spectra. The feasibility of GPU-based NN classification has been widely studied and demonstrated in the literature. Second, atlas factors that affect the computation time may be carefully adjusted (e.g., increase the coherence threshold of the LW-SLSC mask, reduce the number of principal components, reduce the number of acquired frames per wavelength emission) without compromising classification performance. Increasing the coherence threshold would produce smaller masks and thus, less pixels to evaluate for classification. Once a new coherence threshold is de-fined, the number of principal components and frames needed for classification can then be empirically determined by maximizing the combined sensitivity, specificity, and accuracy using Eq. 7. Third, rather than evaluating the photoacoustic spectra with an extensive look-up table (i.e., spectral atlas), the classification time can be reduced by implementing an artificial neural network (ANN) to learn and match features of the acoustic spectra (i.e., bypassing PCA feature extraction), which has been successfully implemented in previous studies (38; 39).

The disclosed framework can be extended to multinomial classification (i.e., more than 2 tissues to classify), which is often necessary during in vivo interventions as photoacoustic signals originating from the surrounding tissue cannot be neglected. In contrast to binomial classification, multinomial classification would benefit from more robust feature extraction methods such as linear discriminant analysis (LDA) or more robust classifiers such as sparse representation-based classifiers (SRC). While the increase in the algorithm complexity can be addressed with the strategies discussed in the previous paragraph, an alternative solution is to include a clustering criteria within the NN classification. Specifically, for trinomial classification where one of the regions would be labelled as a third component that does not exist in the atlas (e.g., background noise). Then, the acoustic spectra from this region can be identified when the error of the closest match surpasses a specific threshold, indicating the presence of a third component. Alternatively, NN-k or fuzzy C-means classification may be employed to characterize the degree of belonging in regions where two materials are combined, with the potential benefit of assessing the percentage of a specific material within a region of interest.

In furtherance of the above disclosure and to further highlight the challenges with traditional spectral unmixing, F-mode, and k-means clustering, the acoustic frequency-based analysis method is discussed to discriminate photoacoustic responses from different materials in comparison to existing approaches. The proposed method does not depend on a reference spectrum (as opposed to k-means clustering). In addition, examples of the present method apply a classification framework using training and testing sets containing known photoacoustic-sensitive materials (i.e., no a priori signal location information is required, unlike F-mode and traditional spectral unmixing techniques). In some examples, the present method, which relies on an analysis of the acoustic frequency response from a single or dual-wavelength emission, is enabled to differentiate biomarkers and has the potential to increase possible frame rates for real-time implementation in the operating room.

To test the method, a frequency analysis was applied to the received photoacoustic signals from two chromophores-blood and methylene blue. The necessity to differentiate these two chromophores is motivated by recently proposed photoacoustic-guided hysterectomy techniques that require differentiation of uterine arteries from ureters containing methylene blue. Although the method is discussed in view of determining the distinction of these two chromophores, the photoacoustic differentiation is applicable to other chromophores of interest during a surgical procedure.

The remainder of this portion of the disclosure is organized as follows. Section 6 details acquisition, segmentation, and classification methods to identify photoacoustic signals originating from either methylene blue or blood, followed by summaries of existing methods used to benchmark the performance of our approach on the same datasets. Section 7 presents the quantitative and qualitative comparison of the classification performance between the proposed and the existing methods. Section 8 discusses insights from these results.

### 6. METHOD

### 6.1 Experimental Setup

A phantom was designed that mimics the clinical setup of photoacoustic catheter-based interventions, where an optical fiber is attached to a cardiac catheter as it is being inserted through a major vein. Another possibility is that a contrast agent may be injected into this vein through the same catheter. Based on these details, FIG. 1 shows the experimental setup used to differentiate the two photoacoustic-sensitive materials of hemoglobin and methylene blue discussed throughout this manuscript. A 29 cm×18 cm×10 cm (length×width×height) polyvinyl chloride-plastisol (PVCP) phantom was fabricated to contain ten cylindrical, hollow chambers. Each chamber had a diameter of 15 mm and a depth of 55 mm. Two of the chambers were filled with either a 100 μM aqueous solution of methylene blue (MB, ID: S25431A, Fisher Scientific, Waltham, MA) or human blood (Hb), and a 1-mm-diameter optical fiber was inserted in each of the filled chambers. These fibers originated from a bifurcated fiber bundle that was connected to a Phocus Mobile laser (Opotek Inc., Carlsbad, CA, USA), transmitting laser light with wavelengths ranging from 690 nm to 950 nm in 10 nm increments.

The tip of each optical fiber was positioned approximately 15 mm below the top surface of the chambers, and light was emitted from each fiber tip. By transmitting light locally into each chamber and not globally illuminating multiple chambers simultaneously, we minimized (or systematically controlled) fluence differences and the related amplitude of responses to the optical excitation. The generated photoacoustic signals were received by an Alpinion L3-8 linear array ultrasound probe (center frequency of 5.5 MHz and pitch of 0.3 mm) that was positioned on the lateral wall of the phantom, approximately 40 mm away from the hollow chamber cross section, as shown in FIG. 1.

To evaluate the reproducibility of our proposed method, three datasets were acquired (i.e., Datasets 1, 2, and 3). Dataset 1 was acquired first followed by Dataset 2 (acquired 10 hours later), followed by Dataset 3 (acquired 13 hours after Dataset 2). The Hb samples were stored with interim refrigeration at a temperature of 4°. The fluence emitted from each fiber tip measured 1.0 mJ.

To characterize the effects of unequal fluence emitted from each fiber tip, three additional datasets were acquired with fluence pairs in the MB and Hb chambers recorded as 0.4 and 1.8 mJ, 1.0 and 1.8 mJ, and 0.4 and 1.0 mJ, respectively. These three datasets were labeled as “Fluence Pair 1”, “Fluence Pair 2”, and “Fluence Pair 3”, respectively. Each fluence pair dataset comprised three subsets, acquired with the same time intervals described in the preceding paragraph for Datasets 1-3.

To evaluate the performance of our proposed method in more challenging environments, particularly in the presence of an aberrating media composed of mostly fatty tissue, three additional datasets were acquired with 2-, 5-, and 7-mm-thick layers of turkey bacon placed between the phantom and the ultrasound probe. This dataset was acquired immediately prior to Dataset 2, thus it is expected to contain similar Hb degradation to that of Dataset 2. The added tissue layers can be considered to represent the fat that is commonly located within skin and within the subcutaneous region of healthy human tissue. The fluence emitted from each fiber tip measured 1.0 mJ.

### 7 RESULTS

### 7.1 Dataset Sensitivity

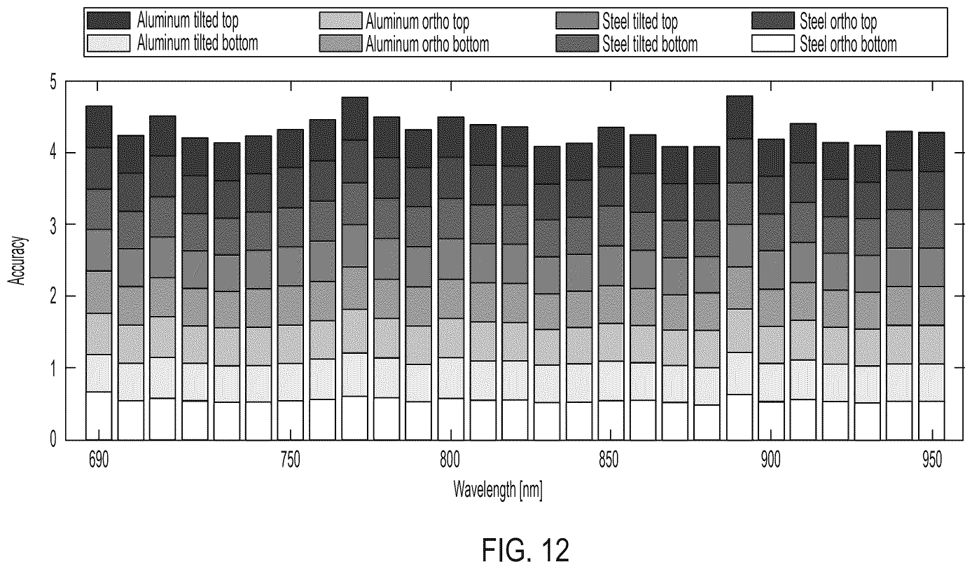

FIG. 13A and FIG. 13B show the accuracy results of the spectral unmixing techniques when tested on Datasets 1-3 while varying the additive coefficient in Eq 3 according to examples of the present disclosure. The dashed line represents the optimal that achieved the highest average f(γ) value among the three datasets. When calculating the optimal 1, 2, and 3 for Datasets 1, 2, and 3, respectively, the absolute difference between the optimal and each 1, 2, and 3 in the conventional spectral unmixing method was 7.01 cm⁻¹, 21.87 cm⁻¹, and 2.70 cm⁻¹, respectively, resulting in a standard deviation of 12.35 cm⁻¹. In contrast, the difference between the optimal γ and the individual γ₁, γ₂, and γ₃ in spectral unmixing+NNMF resulted in a standard deviation of 1.37 cm⁻¹. This γ difference suggests that the optimal of spectral unmixing+NNMF is less sensitive to the testing data than that of conventional spectral unmixing. However, when evaluating the standard deviation of the classification accuracy at the optimal γ, spectral unmixing+NNMF produced standard deviations of 6.47%, 0.54%, and 0.34% for Datasets 1, 2, and 3, respectively, while conventional spectral unmixing produced standard deviations of 1.93%, 0.47%, and 0.48% for Datasets 1, 2, and 3, respectively. Thus, the spectral unmixing techniques did not demonstrate accuracy robustness across different datasets, and this detail is considered in tandem with the γ differences.

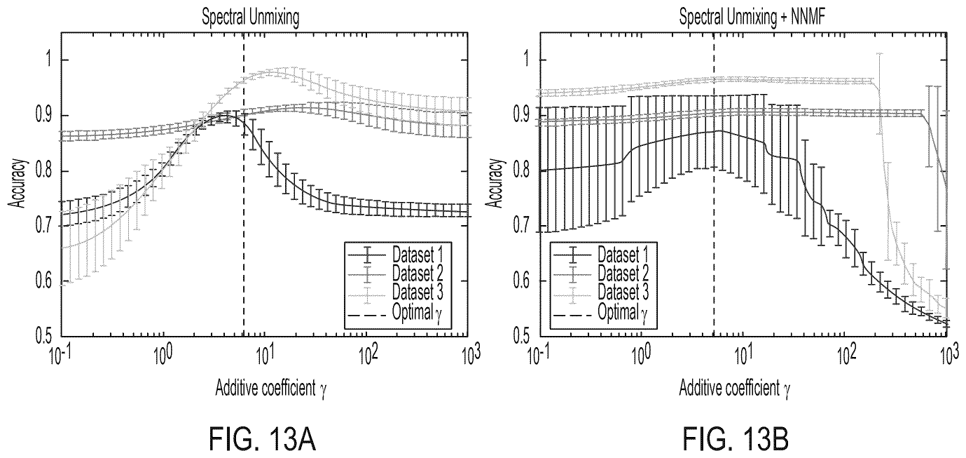

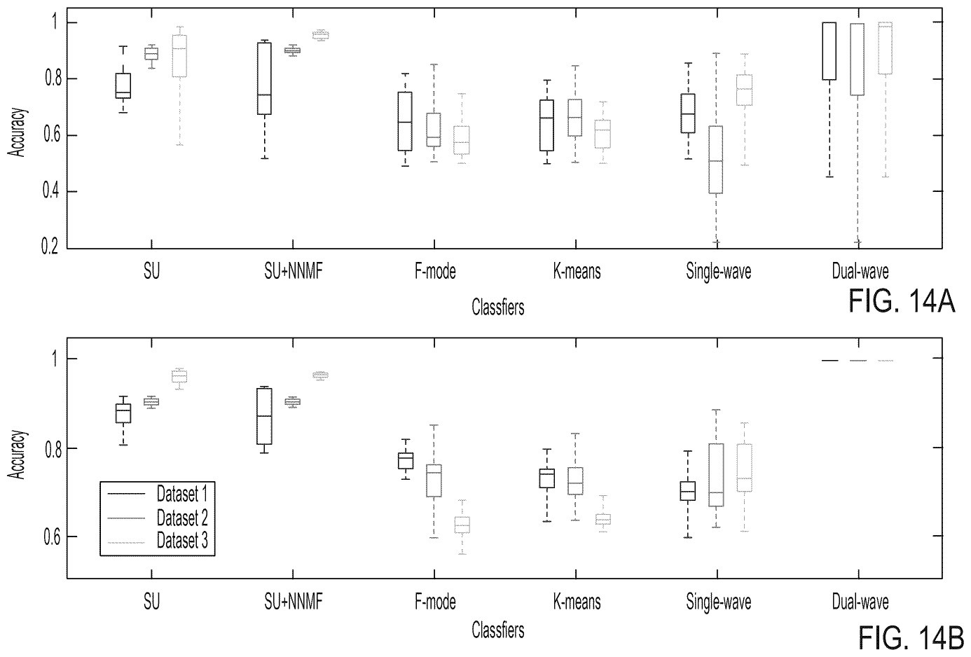

FIG. 14A shows a summary of the accuracy results obtained with the spectral unmixing, single- and dual wavelength atlas, F-mode, and k-means clustering methods using the full range of wavelengths and hyperparameter γ, according to examples of the present disclosure. When comparing the distributions among Datasets 1-3, the maximum difference in median accuracy measured for each dataset when applying spectral unmixing, spectral unmixing+NNMF, F-mode, k-means clustering, and the single-wavelength atlas methods was 15.8%, 21.2%, 6.7%, 4.3%, and 25.5%, respectively, while the dual wavelength atlas method showed a maximum difference in median accuracy of 1.4%. While these results display wide variations due to the inclusion of a wide range of wavelengths, wavelength pairs, or hyperparameter γ, only specific values can be selected in advance and later used in clinical practice.

Therefore, FIG. 14B shows a subset of accuracy distributions obtained from the best 5 cases among the datasets, according to examples of the present disclosure. The dual wavelength atlas method showed a maximum difference in median accuracy between any two datasets of 0%, which was significantly lower than that obtained from spectral unmixing (7.8%), spectral unmixing+NNMF (9.5%), F-mode (15.6%), k-means clustering (10.1%), and the single-wavelength atlas method (3.3%). In addition, when evaluating the dual-wavelength atlas method on Datasets 1-3, the 710-870 nm wavelength pair was present among the 5 pairs of wavelengths with the highest accuracy, both with a sensitivity, specificity, and accuracy of 1.00, respectively. Therefore, the dual-wavelength atlas method implemented with this wavelength pair shows higher reproducibility of classification performance than spectral unmixing and other acoustic-based methods.

### 7.2 Sensitivity Against Fluence Changes

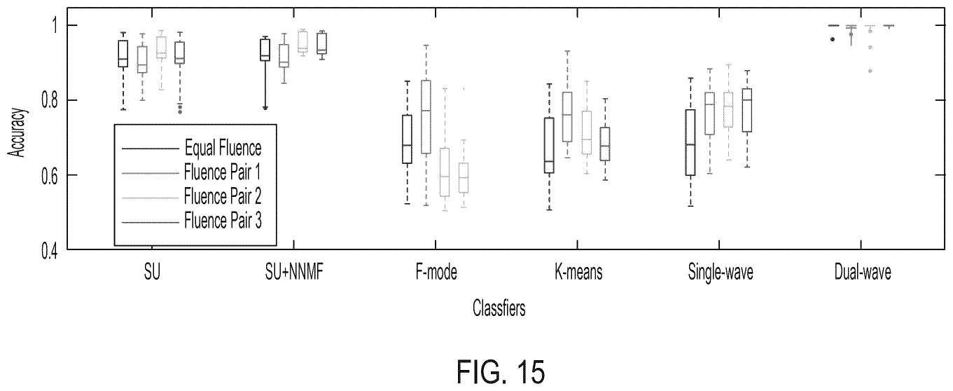

FIG. 15 shows the classification accuracy of each dataset acquired with varying fluence pairs, according to examples of the present disclosure. The best 5 cases of wavelengths and hyperparameter γ. For each of the five existing methods, the maximum difference between the median accuracy values reported for any two fluence pairs (including the “Equal Fluence” Pair) was 3.3% for spectral unmixing, 3.8% for spectral unmixing+NNMF, 18.4% for F-mode, 12.9% for k-means clustering, and 11.9% for the single-wavelength atlas method. For the dual wavelength atlas method, the maximum difference between the median accuracy values reported for any two fluence pairs was 0.1%, which is lower than the values reported for the five existing methods. These results demonstrate that the dual-wavelength atlas method is robust against changes in fluence levels when compared to acoustic-based methods that do not apply a normalization step such as F-mode.

7.3 Performance with Aberrating Media

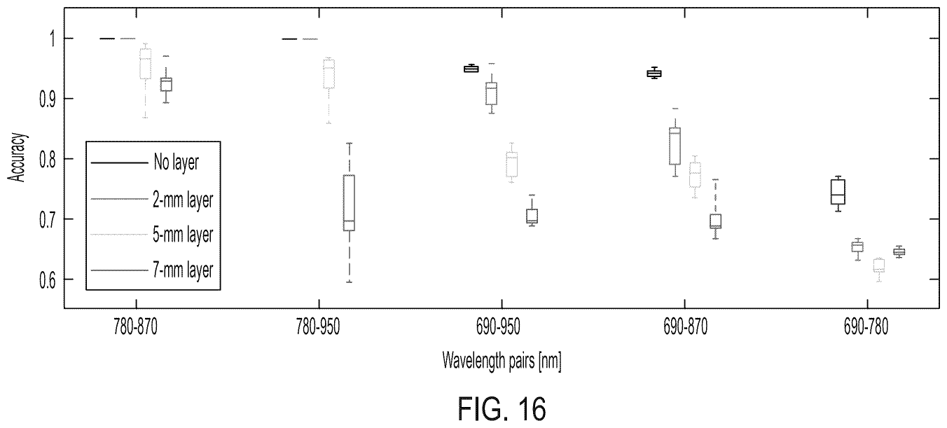

FIG. 16 shows the classification accuracy from the dual-wavelength atlas method tested on the datasets obtained with added tissue layers, according to examples of the present disclosure. The five wavelength pairs are sorted by the median accuracy obtained in the absence of a layer (i.e., in descending order from left to right). When comparing the results obtained in the absence of a tissue layer, the 2-mm tissue layer resulted in no significant change to the median accuracy for the 780-870 nm and 780-950 nm wavelength pairs and decreased median accuracies of 3.0%, 11.9%, and 8.0% for wavelength pairs of 690-950 nm, 690-870 nm, and 690-780 nm, respectively. When comparing the results obtained in the absence of a tissue layer with the 5-mm tissue layer results, the 780-870 nm and 780-950 nm wavelength pair showed a decrease in median accuracy of 3.5% and 4.9%, respectively, which was lower than those obtained with the remaining wavelength pairs, yielding an average median accuracy decrease of 14.4%. Finally, when comparing the results obtained in the absence of a tissue layer with the 7-mm tissue layer results, the 780-870 nm wavelength pair showed a decrease in median accuracy of 7.1%, which was significantly lower than that obtained with the other wavelength pairs, yielding an average median accuracy decrease of 22.5%. Results demonstrate that the aberrating conditions generally reduce performance due to the presence of the fatty tissue, with a median classification accuracy of 92.9% for a 7-mm-thick tissue layer when using a wavelength pair of 780-870 nm.

### 8 DISCUSSION

To maximize the sensitivity, specificity, and accuracy of the disclosed approach, the parameters for in-phase quadrature demodulation, PCA, and NN were first optimized through an iterative search. These parameters were the modulation frequency, filtered bandwidth, axial kernel size, number of principal components to use, and the k nearest neighbors used to determine the most common class in k-NN clustering. Each parameter was changed one at a time, and this search was repeated each time the parameter was changed. During each analysis, the optimal parameter found from the previous step was saved and used to find the new output until the optimal set of parameters were found. For the IQ-modulation, different combinations of bandwidth of 80-240% in intervals of 20% were tested with the modulation frequency which ranged from 1 MHz to 6 MHz in intervals of 1 MHz. Similarly, the axial kernel size was explored from 11 to 51 axial samples in increments of 2. For the PCA, the principal components were changed from 10 to 200 in steps of 10 and then from 1-10 in steps of 1. Finally, the NN classifier was analyzed by changing the classifier to 2-NN through 10-NN in increments of 1. The optimization process was conducted on datasets obtained with laser wavelengths ranging from 690 nm to 950 nm in 10 nm increments.

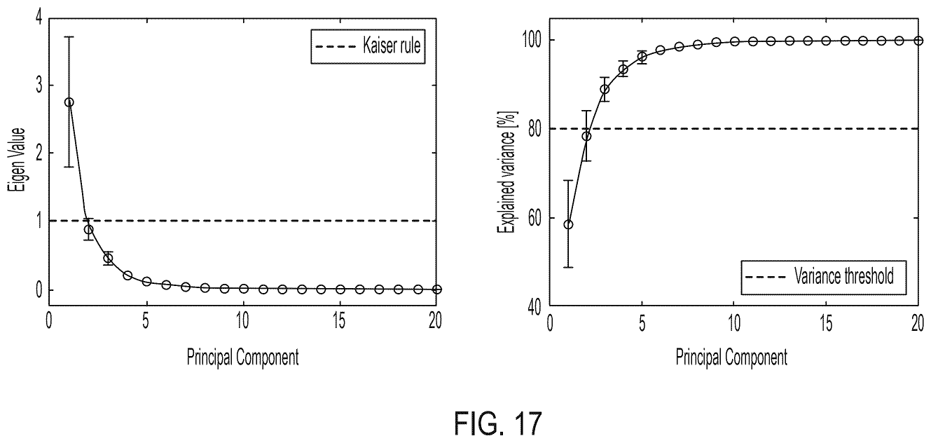

FIG. 17 shows two known criteria to determine the optimal number of principal components used in the dual-wavelength atlas method. Each scatter point and error bar represents the mean and one standard deviation, respectively, of eigenvalues (left plot) and explained variance (right plot) from the 351 wavelength pairs at a specific principal component. For the Kaiser rule (2), only the first principal component shows a distribution with values greater than 1.00, while the second principal component shows a mean eigenvalue less than 1.00. Similarly, the commonly accepted 80%-explained-variance threshold filter the first and second principal component when considering the mean value. However, when considering the standard deviation, the second principal component surpasses the variance threshold. Therefore, only the first principal component was used for the feature extraction in the dual-wavelength atlas method.

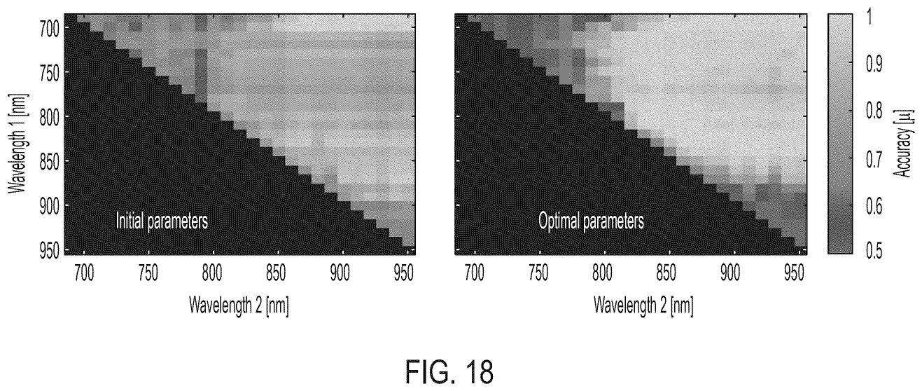

FIG. 18 shows the mean accuracy of classification results for the dual-wavelength atlas method before (left) and after (right) parameter optimization, according to examples of the present disclosure. The top right half of each image represents a triangle with each pixel displaying the average accuracy of classifying methylene blue and blood for a specific wavelength pair over 10 frames. The new parameter set comprises a modulation frequency of 1 MHz, bandwidth of 140%, 30 axial samples, 1 principal component, and 1-NN. For each triangle, the “low-wavelength region” is defined as wavelength pairs <800 nm, the “high-wavelength region” as wavelength pairs >850 nm, and the “mid-wavelength region” as wavelengths that do not belong to either the low- or high-wavelength regions. For wavelength pairs located in the mid-wavelength region, the mean accuracy increased from 85.45% to 95.67%. In contrast, for wavelength pairs that located in either the low- or high wavelength region, the mean accuracy decreased from 74.67% to 67.40%. In clinical practice, a reduced set of wavelength pairs can be used, choosing those that maximize the sensitivity, specificity and accuracy (i.e., mostly occurring in the mid-wavelength region) and otherwise omitting wavelength pairs that result in poor classification performance.

FIG. 17 shows criteria for determining the optimal number of principal components for the dual-wavelength atlas method using the (left) Kaiser rule (2) and (right) a 80% variance threshold, according to examples of the present disclosure.

FIG. 18 shows mean accuracy classification results of the dual-wavelength atlas method per wavelength pair with a dataset of 10 frames per wavelength using the initial parameters of Gonzalez et al. (1) and the updated parameters.

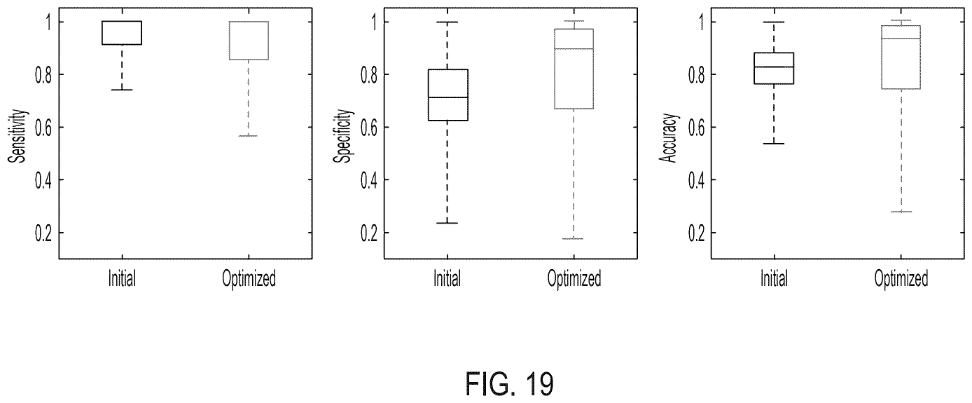

FIG. 19 shows a summary of sensitivity, specificity, and accuracy among the 351 wavelength pairs before and after parameter optimization. The mean±one standard deviation of sensitivity showed no significant change from 91.6±16.8% to 90.9±14.8%. However, an increase in specificity was observed from 70.7±16.9% to 81.7±20.9%, which in turn resulted in an overall increase of specificity from 81.5±10.4% to 85.9±17.1%. Because blood was considered as negative samples for the specificity metric, the results suggest that the optimized version of the dual-wavelength atlas method can identify blood with more accuracy than with the initial parameter set used in Gonzalez et al.

FIG. 19 shows a summary of sensitivity, specificity, and accuracy results of the dual-wavelength atlas method before and after parameter optimization, according to examples of the present disclosure.

FIG. 4 shows the ultrasound acquisition setup used to evaluate the performance variability of the method proposed by Cao et al. (3). In particular, eight ultrasound reference spectra were acquired from two materials: an aluminum plate and a carbon-steel plate. These two materials contain relatively flat, reflective surfaces, which make them ideal for use as reference spectra. These two materials also have acoustic properties that produce different pulse-echo responses, which affects the normalization process applied to the acquired spectra and results in different classification accuracies.

Acquisitions were obtained after independently placing each material at axial depths of 5 cm or 9 cm from the L3-8 ultrasound transducer, tilted 45° or 0° relative to the elevation-lateral plane of the transducer. Each reference spectra was calculated by averaging the fast Fourier transform of scan lines across the lateral dimension. Then, a comparison of the accuracy results was conducted only for the subsets obtained from the optimal laser wavelength λ, defined by Eq. 7 in the manuscript. The reference spectra that produced the highest median classification accuracy was selected for comparison of the method by Cao et al. (3) with other methods reported throughout the manuscript.

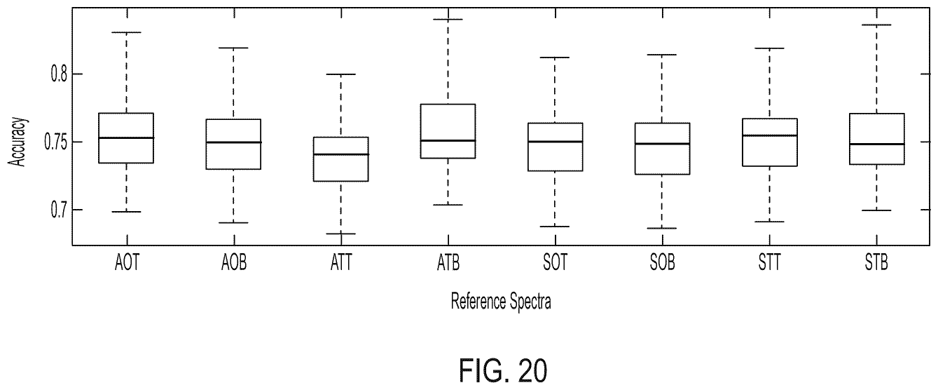

FIG. 20 shows a mean classification accuracy of k-means clustering with different reference spectra, according to examples of the present disclosure. The box plots represent the group of frames evaluated with laser wavelength that achieved the overall best accuracy among reference spectra (i.e., 840 nm). The three letter codes on the abscissa represent the material of the reference (i.e., A=Aluminum or S=Steel), followed by the orientation of the material relative to the ultrasound transducer (i.e., O=Orthogonal or T=Tilted), followed by the imaging depth of 5 cm or 9 cm (i.e., T=Top or B=Bottom, respectively).

FIG. 20 shows the classification accuracy achieved after performing the k-means clustering step of the method of proposed by Cao et al. (3), obtained with a laser wavelength of 890 nm (which was the optimal parameter λ defined by Eq. 7 in the manuscript) when using the reference spectra described above. The reference spectra reported for the k-means clustering method in FIG. 7 of the manuscript was obtained from the tilted aluminum reference with an imaging depth of 9 cm (i.e, ATB in FIG. 20). This reference was chosen because it produced the greatest classification accuracy (i.e., 84.0%).

As discussed above, in the dual wavelength atlas method, an algorithm is used for classification of chromophores that includes building a database of photoacoustic signals originating from chromophores by stacking the acoustic signal response from two different light wavelength emissions. As discussed above, FIG. 3 shows the pipeline for the dual wavelength atlas method according to examples of the present disclosure. After acquiring the photoacoustic signals, the normalized power spectra are calculated from a sliding window of axial kernels of in-phase and quadrature (IQ) data of 3.85 mm in length. These complex data is converted to baseband with a modulation frequency of 2 MHz and a band-pass filter of 140% bandwidth. To reduce the dimensionality of the feature space, principal component analysis (PCA) is applied to the power spectra of photoacoustic signals acquired at each laser wavelength and only the first principal component will be considered for the database. The reasoning for the selection of these parameters is based on the success of the photoacoustic differentiation of dual wavelength atlas method. As was previously found the optimal classification performance was achieved with the laser wavelength pair of 710 nm and 870 nm, the same wavelength pair values are investigated. When using a training set, the principal components are stored in an “atlas” describing each material. Finally, when evaluating the spectra of an unknown signal, nearest neighbor (NN) classification is applied with the L2-norm as the measure of distance between the PCA of the unknown spectra and the atlas.

To characterize the rate of endogenous and exogenous chromophores in a sample, a fuzzy-based clustering method can be used. This method uses the output of the nearest neighbor scheme and estimate the probability of belonging to either exogenous or endogenous chromophore by weighting the residual errors from the N-closest matches. In this example, a compound of 50% indocyanine and 50% blood (i.e., C5) is obtained from the testing set. After running the dual wavelength atlas method, the 7 closest matches of pure blood and indocyanine are shown with their respective residual error when comparing the PCA components of the C5 sample and the training atlas. Then, the errors are transformed to weights, which are proportional to the difference with the 7th closest match. Finally, the summed weights are calculated from the first 6 closest matches, and the percentage of belonging are rounded to the nearest rate of endogenous and exogenous chromophores.

To evaluate the reproducibility of the disclosed classification framework, the training and testing process is repeated for the 3 exogenous chromophores 10 times and compared to the classification performance within the 10 trials. These new training and testing sets are obtained with new contrast agent samples and vials of blood at different time frames. In order to prevent changes of photoacoustic response from blood due to decreased oxygenation levels over time, vials of blood within a time frame no longer than 2 days after acquisition are used, with interim refrigeration storage at a temperature of 4° C. Alternatively, a minimum performance level for the disclosed classifier can be determined when experimenting with each of the exogenous chromophores. If the performance achieved is below the defined threshold, the hyper-parameters used in segmentation and the dual wavelength atlas method can be tuned accordingly and the experiments repeated for each exogenous chromophore. These parameters are not expected to vary significantly relative to the initial parameters selected for the classifier.

In some examples, a mathematical formulation of the photoacoustic frequency response of methylene blue, deoxyhemoglobin, indocyanine green, and intralipid is disclosed. A neural network is trained to predict the photoacoustic response from a random mixture of exogenous and endogenous chromophores using a controllable generative-adversarial network architecture (CGAN). This model can be trained by separating feature classifiers from the discriminator with data augmentation technique, which can be used to make a fine classifier. For each exogenous chromophore, the corresponding testing set is used to train the discriminator and the generator with the goal of mimicking the photoacoustic response of different concentration levels. For this purpose, the corresponding labels L are indexed from 0.0 to 1.0 in increments of 0.1, corresponding to volume percentages of 0% to 100% in increments of 0.1, respectively (see FIG. 6). The CGAN is used in generating photoacoustic response from new concentration levels by using interpolated values of L (i.e., 0.12) which are not trained in the neural network. Alternatively, because one challenge of training a CGAN is the amount of data available in the training set, experimental data can be used for training the network, as opposed to using simulating data. An increase in noise level and artifacts can potentially hinder the performance of both the discriminator and generator in the CGAN. The framework with the exogenous chromophore identified as discussed above as having the highest classification accuracy. If the trained network fails to properly converge and reduce the loss function or is subject to overfitting effects, the network can be retrained with the experimental acquisitions from the second-best exogenous chromophore as training data.

In some examples, a method by estimating the concentration of contrast agent in the cancellous bone of an ex vivo human cadaver is disclosed. With the framework as discussed above, the accuracy of the classifier can be tested when estimating the concentration level of exogenous and endogenous chromophores inside human vertebrae. With the aid of a surgeon, a human cadaver (preferably adult) can be placed in prone position and dissection will be carried along the craniocaudal axis with the aid of a Cobb elevator to reveal the spinous process, lamina, and facet joints at each level from L1 to S1. Prior to the experiment, a CT volume will be acquired to confirm the absence of spine pathologies, malformations, or previous spinal surgeries. The pedicles can be cannulated bilaterally from L1 through L5 along anatomic trajectories using a standard free hand technique with a pedicle probe. Each cannulation will produce hollow cores where the tip of the fiber will be in direct contact with the vertebral body, which is primarily composed of cancellous bone.

A 1-mm diameter optical fiber can be inserted to touch the bottom of the pedicle hole. The optical fiber can be used to transmit 690 nm and 920 nm wavelength laser light from a Phocus Mobile laser (Opotek Inc., Carlsbad, CA, USA) with an energy of 10 mJ at the fiber tip. Photoacoustic signals can be received by a SC1-6 convex array ultrasound probe connected to an Alpinion E-CUBE 12R ultrasound system. The probe can be positioned in an oblique axis across several lumbar laminae. A GPU implementation of SLSC for a convex array can be used to achieve enhanced real-time visualization of photoacoustic signals. This photoacoustic beamforming method can assist the surgeon with fiber tip localization during the surgery. After acquisition of photoacoustic images from the blood-rich structures found in the cancellous bone, a concentration of an exogenous chromophore can be injected into the bottom of the pedicle hole, which will mix with the surrounding blood in the cancellous bone. The spectra of photoacoustic signals are obtained of similar characteristics to those studied as described above. The exogenous chromophore is used that achieves the best classification accuracy. The disclosed classifier can be validated by comparing the estimated volume concentration given by the algorithm (V) with the concentration calculated based on the volume of exogenous chromophores injected and the existing blood in the cancellous bone (\hat{V}). This evaluation will be conducted both at the pixel and region-of-interest level. If V≤\hat{V}, the algorithm will be considered a success. Otherwise, the algorithm will be classified as failing to appropriately determine the ratio of exogenous and endogenous chromophores.

In still furtherance of the above disclosure, a modified version of the previously discussed acoustic-based atlas method is disclosed to estimate concentration levels from a mixture of two photoacoustic-sensitive materials after two laser wavelength emissions. Photoacoustic data were acquired from mixtures of 100 μM MB and either human or porcine blood (Hb) injected in a plastisol phantom, using laser wavelengths of 710 nm and 870 nm. An algorithm to perform linear regression of the acoustic frequency response from an atlas composed of pure concentrations was designed to assess the concentration levels from photoacoustic samples obtained from 11 known MB/Hb volume mixtures. The mean absolute error (MAE), coefficient of determination (i.e., R²), and Spearman's correlation coefficient (i.e., φ between estimated results and ground-truth labels were calculated to assess algorithm performance, linearity, and monotonicity, respectively. The overall MAE, R², and ρ were 12.68%, 0.80, and 0.89, respectively, for the human Hb dataset, and 9.92%, 0.86, and 0.93, respectively, for the porcine Hb dataset. In addition, a similarly linear relationship was observed between the acoustic frequency response at 2.3 MHz and 870 nm laser wavelength and the ground-truth concentrations, with R² and |ρ| values of 0.76 and 0.88, respectively. As discussed further below, contrast agent concentration monitoring is performed with the disclosed approach. The potential for minimal data acquisition times with only two wavelength emissions is advantageous toward real-time implementation in the operating room.

Exogenous chromophores play a critical role in photoacoustic drug delivery, overcoming imaging challenges associated with visualizing low signal amplitudes, small vasculature targets, and deep structures that suffer from optical and acoustic attenuation. Over the past 20 years, exogenous chromophores have been administered to increase signal contrast with beneficial applications in angiogenesis for cancer monitoring, enhanced photoacoustic angiography, lymph node tracers in breast cancer, deep imaging, lymphatic drainage, and brain imaging. Possible contrast agent chromophores include gold nanostructures, carbon nanotubes, fluorescent proteins, methylene blue (MB), and indocyanine green (ICG) dyes.

Depending on the dose, exposure time, and number of target cells, adverse effects due to misuse of contrast agents include acute inflammation, apoptosis, necrosis, cellular toxicity allergies, reduction in cellular viability, nephropathy, hemolysis, and photodamage. In addition, maximum absorption and clearance times (i.e., the time for the drug to completely leave the system) range from 5 minutes to 24 hours among the drug delivery approaches reported in the literature requiring different dose, delivery, and monitoring protocols.

Currently, most approaches to measuring the concentration levels of chromophores rely on the acquisition of photoacoustic responses from multiple laser wavelength emissions paired with spectral unmixing methods. However, as real-time implementations are necessary for monitoring chromophores of short clearance time in applications like photoacoustic-guided surgery, traditional techniques are typically not feasible because of the lengthy acquisition times associated with transmitting multiple laser wavelengths to achieve a single estimate. In addition, traditional spectral unmixing techniques do not typically consider differences in acoustic spectra, which has the potential to provide additional information for differentiation of exogenous and endogenous chromophores.

As discussed above, the acoustic frequency-based method is used to discriminate photoacoustic responses from different materials and overcome challenges with traditional spectral unmixing techniques. By measuring the photoacoustic response from only two laser wavelengths, the initial version of our dual-wavelength atlas method achieved comparable or better sensitivity, specificity, and accuracy to traditional spectral unmixing methods and related classifiers. Based on the linear relationship between contrast agent concentration and photoacoustic amplitude, the previously discussed method is extended to locally characterize the volumetric ratio between two photoacoustic-sensitive materials. Specifically, the exogenous chromophore MB and the endogenous chromophore hemoglobin (Hb) were investigated, motivated by the potential for photoacoustic based catheter interventions performed with an optical fiber housed within a cardiac catheter inserted through a major vein. MB would be administered through the catheter and eventually mix with Hb to enhance the visualization of target structures in vascular pathologies (e.g., atheroscle rositic plaques, thrombi, tumor neovasculatures, endothelia), and the local concentration of MB vs. Hb would be monitored with the disclosed method with either external or intravascular ultrasound sensor placement. This monitoring will enable real-time interventions to avoid adverse effects produced by the unnecessary accumulation of MB. In addition, the MB used in the present study serves as a surrogate for other potential intravascular contrast agents that would similarly benefit from local concentration monitoring to prevent cell toxicity.

The remainder of this section is organized as follows. Section 9 details the phantom experimental setup, a dual-wavelength atlas method that is used to estimate MB and Hb concentrations, and quantitative metrics for performance evaluation. Section 10 presents the quantitative evaluation of mixture estimation performance, as well as evaluation of linear and monotonic trends of estimated concentration versus ground-truth labels, including an assessment of appropriate region sizes to perform the disclosed estimation. Section 11 discusses insights from these results, and Section 12 summarizes the clinical impact of the disclosed methods.

### 9 METHOD

### 9.1 Experimental Setup

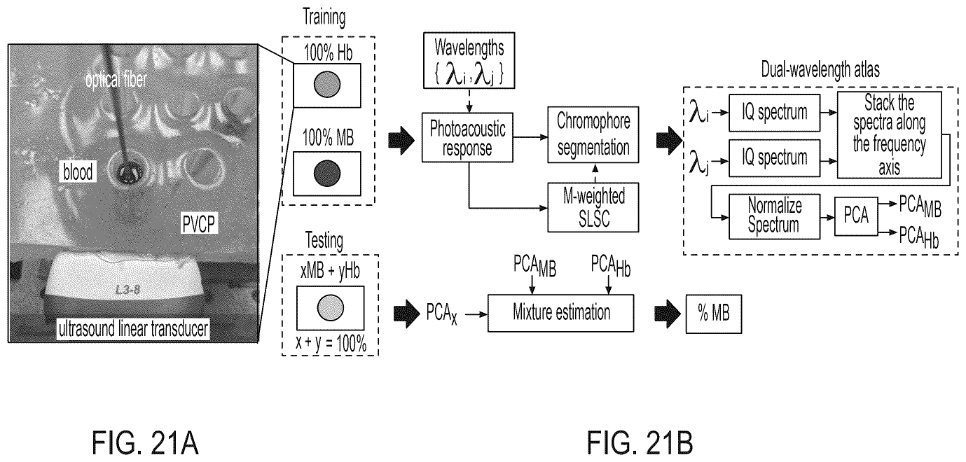

A polyvinyl chloride-plastisol (PVCP) phantom was fabricated with hollow chambers, each with a diameter of 15 mm and a depth of 55 mm. To minimize the variability of ultrasound scattering among chambers due to different air bubble distributions, a single chamber was filled with either a 100 μM aqueous solution of MB from Fisher Scientific (Waltham, MA), blood (Hb), or a combination of MB and Hb. A 1-mm-diameter optical fiber was inserted in the filled chamber, and the fiber tip was positioned approximately 20 mm below the top surface. The optical fiber was connected to a Phocus Mobile laser (Opotek Inc., Carlsbad, CA, USA), transmitting laser light with wavelengths of 710 nm and 870 nm and with a laser energy of 3 mJ. The resulting photoacoustic signals were received by an Alpinion L3-8 linear array ultrasound probe (Alpinion Medical Systems, Seoul, South Korea) positioned on the lateral wall of the phantom, approximately 20 mm from the hollow chamber cross section. This experimental setup, which is shown in FIG. 21A, has been described in previous publications.

FIG. 21A show an experimental setup and FIG. 21B shows a framework of the dual-wavelength atlas method for mixture estimation of methylene blue (MB) and Hb (Hb).

Two types of Hb samples were used in this study. First, 23 vials of fresh human Hb were obtained up to two days after blood draw and storage, mixed in a single container, and exposed to air for approximately 1 hour to minimize the variability of Hb oxygenation levels. Second, experimental whole porcine blood (Innovative Research, Novi, MI) was used on the fourth day of its reported 24-day lifetime.

A total of 11 concentration levels were prepared using different 2 mL mixtures of Hb and MB. These concentrations ranged from 0% to 100% in 10% increments of MB volume percentage. For example, for the 2 mL MB-Hb mixture, a concentration of 60% MB consisted of 1.2 mL of 100 μM MB and 0.8 mL of Hb. Each concentration mixture was manually stirred with a syringe in a separate container. Unless otherwise stated, the % concentrations reported in this manuscript refer to the % MB concentration. Five trials were conducted for each concentration level by fixing the light-delivering optical fiber at 0°, with 20 photoacoustic frames (10 per wavelength) acquired per trial using a frame rate of 5 Hz. Note that two blocks of 64-channel aperture data were required to obtain an image created from 128 receive elements, which reduced the possible frame rate from 10 Hz (based on the 10 Hz laser pulse repetition frequency) to 5 Hz.

### 9.2 Dual-Wavelength Atlas Method for Mixture Estimation

The framework for mixture estimation is illustrated in FIG. 21B, based on the dual-wavelength atlas method previously discussed for the identification of individual chromophores. With this setup, frequency domain information is expected to be useful because the optical fiber is placed inside the PVCP chamber, thus generating fluence maps that diminish radially from the tip of the optical fiber. This fluence distribution generates unequal acoustic frequency response, as volume regions of different sizes are being excited. Thus, the disclosed algorithm is anticipated to leverage frequency domain information to differentiate photoacoustic responses from various concentrations of chromophores.

To implement the present approach, conventional delay-and-sum (DAS) beamforming was first employed to create photoacoustic images for each wavelength emission, concentration, and trial. In contrast to preceding work, a M-Weighted short-lag spatial coherence (SLSC) with a cumulative lag M=20 is used instead of conventional SLSC with M=5 to generate the binary masks that segmented signals of interest and provided ground-truth labels. This specific M-Weighted SLSC cumulative lag value (i.e., M=20) was chosen based on the comparison of SLSC images with M-weighted SLSC. First, a representative SLSC photoacoustic image from the blood dataset was generated with M=5. The same photoacoustic frame was then processed with M-weighted SLSC with M values ranging from 10 to 30. Binary masks generated with a −3 dB threshold were then created for each image, and the similarity was defined with the Dice coefficient:

$$Dice Coefficient = \frac{2 \sum AND ( {Mask}_{SLSC} , {Mask}_{M - Weighted SLSC} )}{\sum {Mask}_{SLSC} + \sum {Mask}_{M - Weighted SLSC}} \times 100 \% , \qquad ( 8 )$$

where AND is the logical “AND” operator between the binary masks. This process was then repeated for one trial of the human Hb dataset, containing 11 concentrations, 2 wavelengths, and 10 frames. The Dice coefficients calculated between SLSC and M-Weighted SLSC masks were then plotted as a function of M, resulting in M=20 yielding the highest mean similarity to the SLSC masks with the least standard deviation.

The modification from SLSC in our preceding work to M-Weighted SLSC in this work was implemented to increase the influence of lower lags over higher lags, thus creating less disjointed binary masks. A single binary mask was obtained per trial, resulting from the logical inclusive “OR” operation of 20 masks (obtained from 10 frames per two laser wavelengths).

As noted in FIG. 21B and in our previous work, for each image, only those pixels included in the coherence masks were used for feature extraction, training, and classification. In-phase and quadrature (IQ) demodulation was implemented with 2.75 MHz and 85% bandwidth, and the resulting analytic spectra from a sliding window of axial kernels was stacked along the frequency axis. For the last step of feature extraction, principal component analysis (PCA) was applied to the power of the stacked spectra in order to reduce the complexity of the feature space.

The training dataset comprises a random trial of 0% MB (i.e., 100% Hb) and 100% MB, and the remaining trials and concentration levels were included in the testing datasets. After the dual-wavelength atlas was constructed (i.e., PCA_{MB} and PCA_{Hb} in FIG. 21B), a mixture estimation algorithm was designed to obtain the concentration level of MB from a testing sample. First, N=1, 000 samples were randomly selected from the MB and Hb atlas. Then, assuming that mixtures are linear combinations of pure MB and Hb concentrations, a concentration distribution C″ ∈ [symbol] ^{N×1} was obtained using the following equation:

$${C}_{x}^{′} = \frac{1}{2} + \frac{{‖ {PCA}_{x} - {PCA}_{Hb} ‖}_{1} - {‖ {PCA}_{x} - {PCA}_{MB} ‖}_{1}}{2 {‖ {PCA}_{Hb} - {PCA}_{MB} ‖}_{1}} , 0 \leq {C}^{′} \leq 1 \qquad ( 9 )$$

where PCAₓ is the projected spectra of x % testing concentration, ∥·∥₁ is the Manhattan norm, and each PCA matrix is of size [symbol] ^{N×p} with p principal components. A histogram filter was then applied to the C′ vector, where the bin with the highest number of individuals out of 10 bins was extracted. The mean value C″ was computed from the reduced C′ vector, and the process was repeated for each valid pixel and frame of the trial. Finally, a median filter was applied to the concentration maps with a kernel of 0.64×0.70 mm×30 in the axial, lateral, and frame dimensions, respectively. The 0.64 mm axial kernel length was selected to reside within the frequency response of received photoacoustic signals, which ranged 2.00 MHz to 2.75 MHz, corresponding to wavelengths ranging 0.77 mm to 0.56 mm, respectively. As the lateral and axial resolution of an image differs, the lateral kernel width (i.e., 0.70 mm) was chosen to be the closest possible match to the axial kernel length. The frame dimension of the kernel reduced the concentration tensor from C″∈ [symbol] ^{N}^{ᵢ}^{×N}^{ⱼ}^{×N}^{_{f}} to C∈ [symbol] ^{N}^{ᵢ}^{×N}^{ⱼ}, where Nᵢ and Nⱼ are the number of rows and columns, respectively, of the reconstructed photoacoustic image.

Because Eq (9) is applied on frequency data, the three most influential parameters of the dual wavelength atlas method for the estimation of C are the axial kernel size, threshold of spectral log compression, and number of principal components. An increase in axial kernel size improves the accuracy of the estimation of spectral amplitudes in the Fourier space. The threshold of spectral log compression adds a tolerance to the discrimination between spectral peaks, which affects the variance that is computed in the PCA. The number of principal components controls the amount of information that is reduced to the feature space.

To provide complementary information regarding the location of photoacoustic targets in the imaging plane, the displayed concentration maps were overlayed with an ultrasound image of the plastisol chamber processed with locally-weighted SLSC (LW-SLSC), using a regularization factor α=1 and a 1.25 mm×1.2 mm kernel. The LW-SLSC parameters were obtained from our previous optimization study. A factor of α=1 corresponded to an equal weight between cost and penalty function for calculating the optimal coefficients in LW-SLSC. An axial kernel size of 1.25 corresponded to approximately 4.52 where λ is the wavelength associated with the center frequency of the L3-8 ultrasound probe.

### 9.3 Quantitative Metrics

To evaluate the overall accuracy of the dual-wavelength atlas method for mixture estimation, the estimated concentration levels C were first ordered as a function of the ground-truth labels k. The function ƒ₁: ((k) was then compared to ƒ₂: C=k, representing a 1:1 relationship between estimated and true concentrations. Finally, the coefficient of determination R² was used to quantify the performance of the dual-wavelength atlas method:

$${R}^{2} = 1 - \frac{{\sum }_{k} {\sum }_{i} {C}_{ki} - k}{{\sum }_{k} {\sum }_{i} {C}_{ki} - \overline{C}} , \qquad ( 10 )$$

where Cₖᵢ is the estimated concentration of measurement i for a ground-truth concentration k and C̅ is the mean estimated concentration. In addition, the R² metric was used to assess the linearity of the photoacoustic spectra obtained from mixtures of MB and Hb by implementing linear regression of spectral amplitudes as a function of the ground-truth concentrations.

Similarly, monotonicity was evaluated in two cases: (1) between photoacoustic spectral amplitudes of a specific frequency and ground-truth concentrations and (2) between estimated and ground-truth concentrations. The monotonic trend of these data pairs were quantified with the Spearman's rank correlation coefficient ρ, described in the following equation:

$$ρ = \frac{cov ( R ( X ) , R ( Y ) )}{{σ}_{R ( X )} {σ}_{R ( Y )}} \qquad ( 11 )$$

where R(X) and R(Y) are the rank variables of the measurements X and Y, respectively, X is either the spectral amplitudes or estimated concentrations, Y is the ground-truth concentration, cov represents covariance, and σ is the standard deviation of the rank variables. Spearman's ρ of values 1.0 and −1.0 represent perfect monotonic trends between measurements X and Y that are increasing and decreasing, respectively. Note that the Spearman's ρ measures the level of monotonicity, rather than determining whether or not an evaluated function is monotonic. In this example, |ρ|≥0.8 is characterized as a strong monotonic trend.

The mean absolute error (MAE) was used to assess the accuracy of the generated concentration maps:

$${MAE}_{k} = \frac{{\sum }_{i = 1}^{{N}_{k}} \vert {C}_{ki} - k \vert}{{N}_{k}} \qquad ( 12 )$$

where Cₖᵢ is the estimated concentration of pixel i for a ground-truth concentration k, and Nₖ is the total number of pixels of the concentration map k.

Finally, processing times were computed for each stage of the dual-wavelength atlas method applied on the human Hb dataset. These stages were: (1) generation of coherence masks, (2) generation of acoustic spectra and spectra stacking, (3) PCA, and (4) estimation of MB concentrations, where the last stage included projection to the feature space using the principal component coefficient obtained from the atlas. These processing times were evaluated individually when training our method (i.e., constructing the atlas) and testing our method on a single frame (i.e., estimating a concentration map). The processing times for Stages (3) and (4) were only calculated for training and testing, respectively. For training, robustness in the estimation of computation times was achieved by averaging the processing times from 5 different trials of 0% concentration. Similarly, robustness in the estimation of computation times for testing was achieved by averaging the processing times from every frame of the testing dataset detailed above. Computation times were measured using the MATLAB (Natick, MA) environment executed on an Intel Core i5-218 6600K processor with 32 GB RAM and a TITAN Xp graphical processing unit (GPU). To enhance the computation speed of Stage (1) for all results reported in this manuscript, M-weighted SLSC was implemented on the GPU described above, following the architecture of preceding work. In addition, this GPU-M-Weighted SLSC approach and the previously-reported GPU-SLSC approach were both implemented to obtain results for Equation 1 in Section 9.2.

### 10 Results

10.1 Concentration Estimations from MB and Human Hb

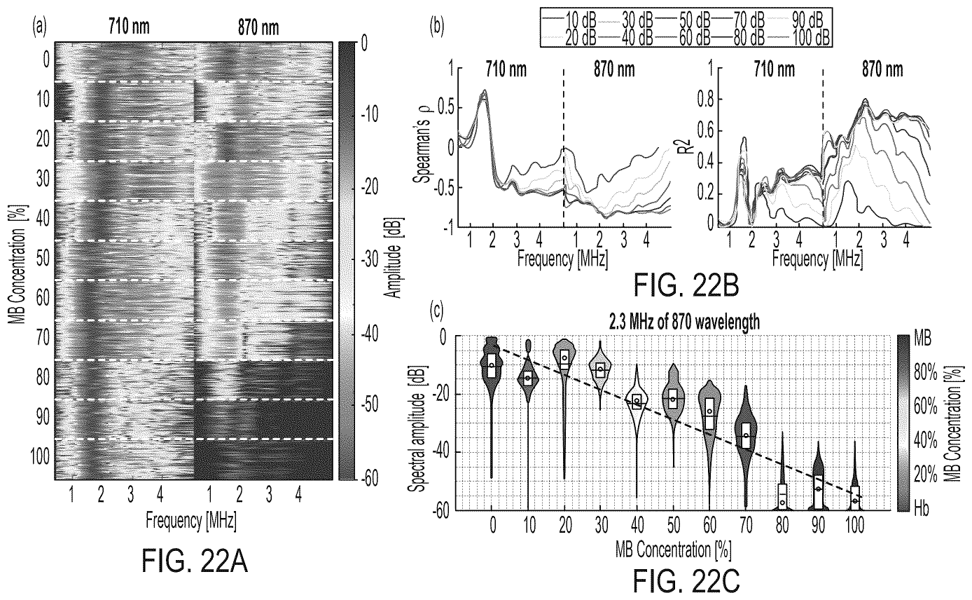

FIG. 22A, FIG. 22B, and FIG. 22C show the results from the photoacoustic spectra obtained with mixtures of MB and human Hb. Stacked photoacoustic spectra are shown in FIG. 22A with the y axis denoting mixture concentration and the x axis denoting acoustic frequency for optical wavelengths 710 nm (left) and 870 nm (right). Each concentration block separated by the dashed white lines in FIG. 22A shows 20,000 spectra samples that were randomly selected from the total number of training trials, frames, and 230 kernels. Although a subtle frequency shift is observed in the spectra obtained with the 710 nm wavelength when increasing the MB concentration level, the spectral shift across the mixture for the 710-nm response was not strongly monotonic (ρ=−0.63). Similarly, no apparent spectral shift was observed for the 870-nm response (ρ=−0.27). One possible cause of the frequency shift in the spectra obtained with the 710 nm wavelength is the photoacoustic interaction with residual particles of MB in the PVCP chamber. These particles were unable to be removed as they stained the chamber walls and they likely added frequency components to the overall spectral response of different chromophore concentrations.

FIG. 22B displays the linearity and monotonicity evaluation results as a function of the acoustic frequency for multiple image dynamic ranges. The y axes denote the ρ (left) and R² (right) for and linearity and monotonicity evaluations, respectively. The x axis of each plot is divided into acoustic frequencies for optical wavelengths of 710 nm (left) and 870 nm (right). In the acoustic frequency range 0.5-5 MHz for 870 nm wavelength, as the dynamic range decreases from 40 dB to 10 dB, the mean±one standard deviation of improvements in Spearman's ρ and R² were −0.47±0.15 and 0.45±0.14, respectively. For dynamic ranges greater than 40 dB, the results primarily overlap, and there is less improvement. This overlap likely occurs because the log compression reaches the resolution limit of the acquired photoacoustic amplitudes. However, for 710 nm wavelength, only the acoustic frequency range from 2.4-5 MHz shows ρ and R² changes when varying the log compression from 10 to 40 dB. In the acoustic frequency range 0.5-2.4 MHZ, results primarily overlap regardless of the chosen dynamic range. This overlap likely occurs because of the strong spectral peak observed from 1.5 MHz to 2.5 MHz for nearly every MB concentration in FIG. 22A (i.e., the spectral shift pattern described in the preceding paragraph). This strong spectral peak is robust against log compression, which generates minimal changes to the Spearman's ρ and variation of to the R².

FIG. 22A, FIG. 22B, and FIG. 22C show evaluation of monotonicity and linearity of photoacoustic spectra obtained from mixtures of MB and Hb. (FIG. 22A) Stacked photoacoustic spectra of several mixture concentration (y axis) when using 710 nm and 870 nm laser wavelength (x axis). Each spectrum is normalized and log compressed to a 60 dB dynamic range. (FIG. 22B) Spearman's ρ coefficient and R² values of linear regression of concentration levels vs. acoustic frequency while varying the log compression dynamic range of the acoustic spectra. (FIG. 22C) Example of an acoustic frequency whose spectral amplitude of concentration levels follow a monotonic trend (ρ=−0.88) and a linear trend (R²=0.76).

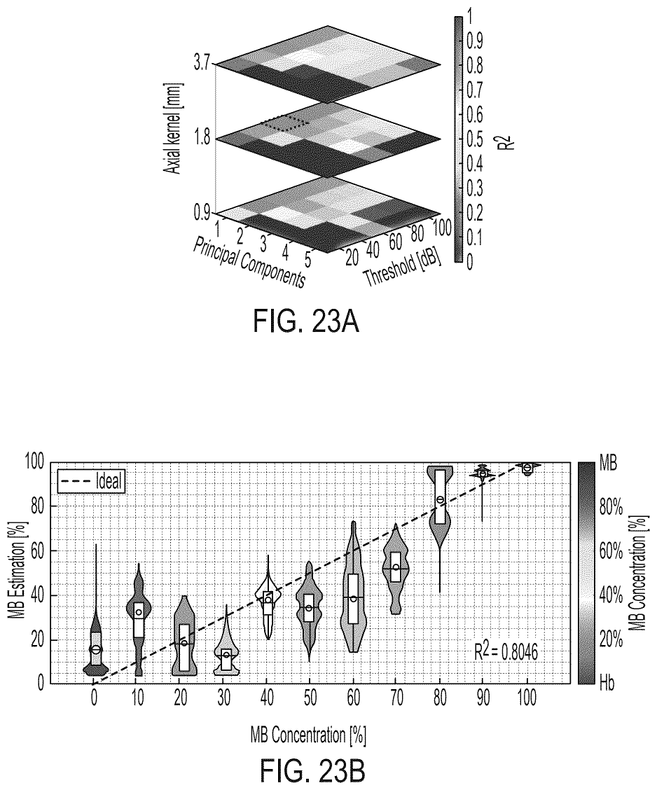

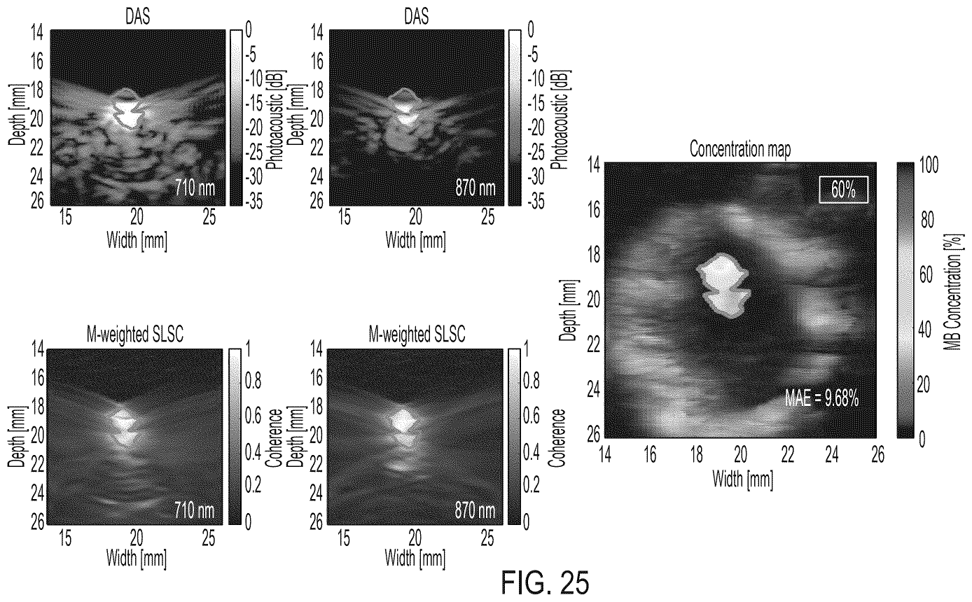

FIG. 23A and FIG. 23B show performance optimization of the dual-wavelength atlas algorithm using the human Hb dataset. (FIG. 25A) R² values as a function of axial kernel size (z axis), threshold for spectra log compression (x axis), and number of principal components used (y axis). (FIG. 23B) Examples of mixture estimations (y axis) vs ground truth concentrations (x axis) when using the parameter set that yielded the highest R² value (i.e., 0.80), represented by dotted square in (FIG. 23A).

Focusing on the R² results, multiple strong linear trends were observed when results were obtained with 870 nm laser wavelength, yielding a mean±one standard deviation R² of 0.74±0.14 for frequencies ranging from 0.5 to 5 MHz and thresholds from 10 to 100 dB. In contrast, no strong linearity was observed for the 710-nm region, yielding an R²=0.32±0.21 for the same range of frequencies and thresholds as the 870-nm region. The strong spectral peak observed in FIG. 22B at 710 nm wavelength yielded a maximum R² of 0.55 with a dynamic range of 10 dB, while the maximum peak observed in 870 nm was 0.81 with a dynamic range of 50 dB. These results suggest that Hb has a stronger influence on the linear relationship of acoustic frequency vs. concentration compared to MB.

FIG. 22C shows the linear fit for the acoustic frequency of 2.3 MHz and the dynamic range of 60 dB (i.e., parameters that produced the maximum R² in FIG. 22B). The y axis denotes the log-compressed spectral amplitudes, and the x axis denotes the ground-truth concentrations. For each violin plot, the shape, horizontal line, white circle, and gray box represent the kernel density estimate of the data points, mean, median, and interquartile range, respectively. R²=0.78, suggesting a strong linearity and monotonicity between the spectral amplitudes of photoacoustic signals and ground-truth concentrations. The Spearman's p was 0.89, which indicates that there is a high level of monotonicity.

FIG. 23A and FIG. 23B show the optimization of the dual-wavelength atlas method using the human Hb dataset by maximizing the R² fit of a 1:1 slope of estimated vs. ground-true concentrations. The variation of R² values as a function the three most influential parameter of the dual-wavelength atlas method is shown in FIG. 23A. The z axis denotes the axial kernel size, and the orthogonal axes denotes the number of principal components used and threshold for spectra log compression, respectively.

Although increasing the axial kernel size improved the accuracy of the spectral amplitude generated with fast Fourier transforms, no clear improvement was observed when measuring the overall R² values. Similarly, parameter sets using two principal components yielded R²≥0.7, suggesting a good estimation performance when using more than one principal component. However, no improvement was observed for PC≥3. Finally, increasing the threshold from 20 dB to 60 dB improved the R² values when other parameters remained fixed, and no additional improvement was observed for dynamic range thresholds ≥60 dB.

FIG. 23B shows an example of concentration estimation vs. ground-truth concentration when using the optimal set of parameters that yielded the greatest R² value, denoted by the dashed black box in FIG. 23A. For this optimized estimation result, the R² value and Spearman's ρ was 0.80 and 0.89, respectively, indicating a high degree of linearity and monotonicity, respectively.

10.2 Concentration Estimations from MB and Porcine Hb

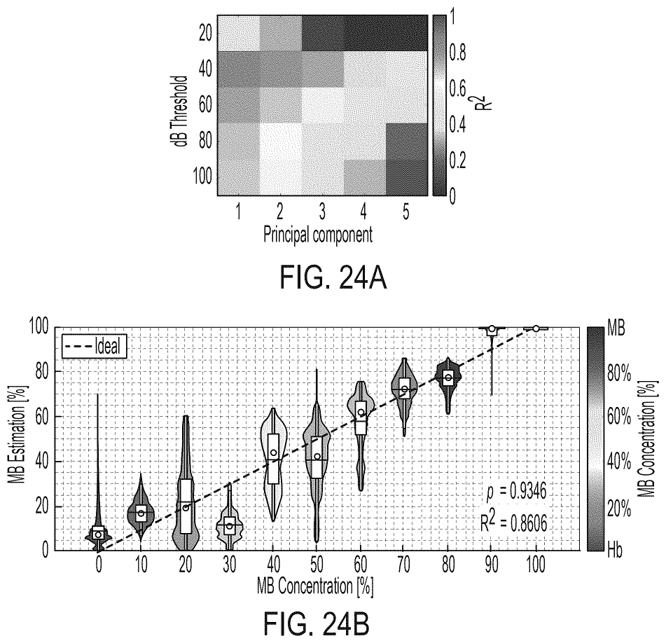

FIG. 24A and FIG. 24B show the estimated concentrations when using porcine Hb. The sensitivity of estimated R² values while varying two parameters of the dual-wavelength atlas method is shown in FIG. 24A with the y-axis denoting the threshold for spectra log compression and the x-axis denoting the number of principal components used. These R² values were computed by fixing the axial kernel size to 1.8 mm (i.e., optimal parameter obtained in FIG. 23A). Overall, the effect of varying the number of principal components and the dB threshold implemented prior to the estimating R² values showed similar trends to those observed in FIG. 23A (i.e., decreasing estimation performance with increasing the number of principal components). However, the optimal set of parameters included a threshold of 40 dB instead of the 60 dB threshold obtained in the experiment using human Hb (i.e., FIG. 23A), with R²=0.86 and ρ=0.93.

FIG. 24B shows detailed estimation results when using the optimal set of parameters from FIG. 24A. The overall performance of the dual-wavelength atlas method when using the porcine Hb was measured by computing the MAE between the vector constructed with the mean of each violin plot and the ground-truth labels, yielding a value of 6.53%. For comparison, the MAE obtained from the optimal parameter set using the human Hb (i.e., FIG. 23B) was 10.49%. Thus, the dual-wavelength atlas method generated more accurate estimates of MB concentration when using porcine Hb instead of human Hb, which can be attributed to the uniformity in chemical composition and oxygenation levels of the porcine Hb samples.

FIG. 24A and FIG. 24B show estimated concentrations from porcine Hb. (FIG. 24A) R² value of a 1:1 linear fit between estimated and true concentration levels while varying the threshold for spectra log compression (y axis) and number of principal components used (x axis). (FIG. 24B) Mixture estimation (y axis) vs ground truth concentration (x axis) when using a 40 dB threshold and 1 principal component.

FIG. 25 shows examples of photoacoustic DAS images, coherence masks used for segmentation, and estimated concentration map for a ground-truth concentration of 60% (i.e., 60% MB and 40% experimental porcine Hb) according to examples of the present disclosure. The purple contours represent the masks obtained by thresholding the coherence images at −3 dB and merging the results from 710 nm and 870 nm.

FIG. 25 shows example DAS photoacoustic images, coherence masks, and an estimated concentration map for a ground-truth concentration of 60% of a single trial. These DAS images were normalized to the maximum amplitude value obtained from both 710-nm and 870-nm laser wavelength responses and displayed with 35 dB dynamic range to offer a direct comparison across wavelengths. The contours within each image represent the −3 dB boundary of the coherence mask with an area of 5.32 mm². Note that these masks include the low-amplitude regions of the photoacoustic images which appear as if minimal signal is present, which highlights the benefit of the coherence mask that is used to determine the presence of coherent signals, regardless of amplitude. The MAE between the concentration map and the ground truth is 9.68%.

Table 5 reports the average processing times for each stage of the dual-wavelength atlas method using the porcine Hb dataset. Without considering acquisition times and transferring times of raw photoacoustic data, the construction of the dual-wavelength atlas was completed in less than 6 minutes, while the mean±one standard deviation processing time of a single concentration map was 9.2±0.5 seconds. This <10 minutes processing time conducted during pre-surgery is relatively fast in comparison to the total procedure times of cardiovascular interventions (e.g., the average procedure time of perfusion coronary interventions is 76±31 min), and it can be performed prior to the initiation of the procedure.

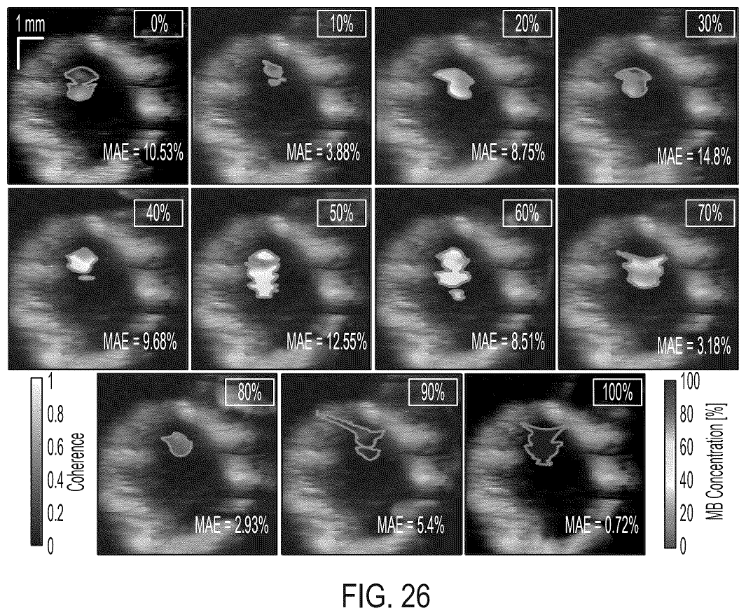

FIG. 26 shows examples of concentration maps obtained from one trial per ground-truth concentration results. The 30% concentration map produced the greatest MAE of 14.80%, whereas the 100% concentration maps produced the lowest MAE of 0.72%. These deviations agree with the overall errors shown in FIG. 24B, where 30% and 100% ground-truth concentrations reported MAEs of 18.29% and 0.75%, respectively. Overall, the dual-wavelength atlas method achieved greater accuracy when estimating concentration levels that primarily include MB.

FIG. 26 shows examples of concentration maps of MB and porcine Hb generated by the dual-wavelength atlas method in a phantom experiment according to examples of the present disclosure. The concentration maps are overlaid on ultrasound images generated with LW-SLSC.

The initial dual-wavelength atlas method used LW-SLSC beamforming to extract meaningful photoacoustic data. The method herein uses M-Weighted SLSC beamforming, rather than LW-SLSC beamforming, in order to reduce the extensive processing times required to generate coherence masks for a total of 2,200 frames (i.e., 11 concentrations×2 wavelengths×10 frames 332×5 trials×2 datasets) of photoacoustic data. However, considering that only a single ultrasound image is needed to show the structural detail surrounding the concentration maps in FIG. 25 and FIG. 26, LW-SLSC was employed to beamform the background ultrasound image and improve the contrast, edges, and interpretation of the structural details surrounding the photoacoustic signals.

### 10.3 Effect of Mask Size on Concentration Estimation Performance

To illustrate the impact of mask size on estimation performance, coherence masks were generated from M-weighted SLSC images with coherence thresholds ranging from 0.3 to 0.9 in increments of 0.02. These masks were used to segment photoacoustic signals from the human blood dataset and generated concentration maps of different sizes. Then, for each coherence threshold, the mean absolute error was calculated for the entire 10 frames per 11 concentration levels.

TABLE 5
Processing times (in seconds) for each training and
testing stage of the dual-wavelength atlas method

| | | Testing (one |
|---|---|---|
| Stage | Training | frame) |
| Generate coherence masks | 135.5 ± 0.4 s | 2.3 ± 0.1 s |
| Convert to frequency | 180.9 ± 23.6 s | 6.4 ± 0.8 s |
| domain | | |
| Apply PCA | 2.6 ± 0.1 s | — |
| Estimate concentrations | — | 0.6 ± 0.2 s |
| Total | 319.2 ± 23.7 s | 9.2 ± 0.5 s |

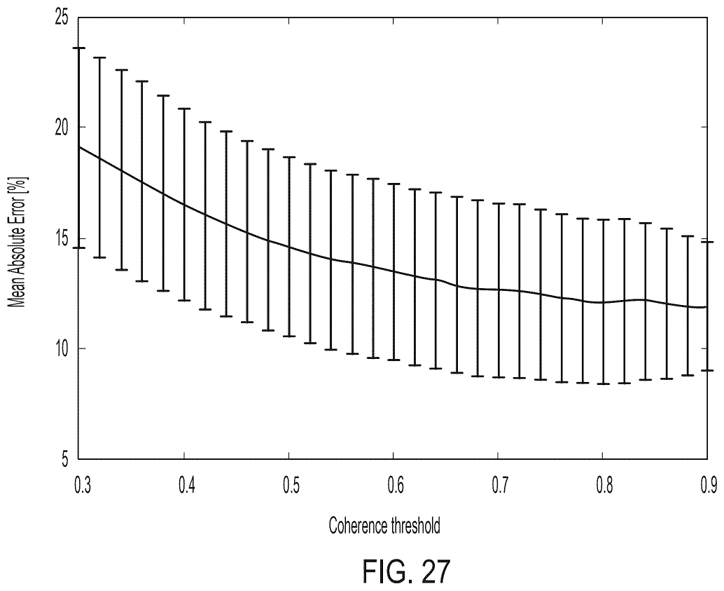

FIG. 27 shows the performance of the dual-wavelength atlas method when using segmented masks generated with varying coherence thresholds. Overall, the mean absolute error decreases when using higher coherence thresholds to generate the segmentation masks. Therefore, a coherence threshold of 0.7 is selected to generate all coherence masks in the experiment results of manuscript.

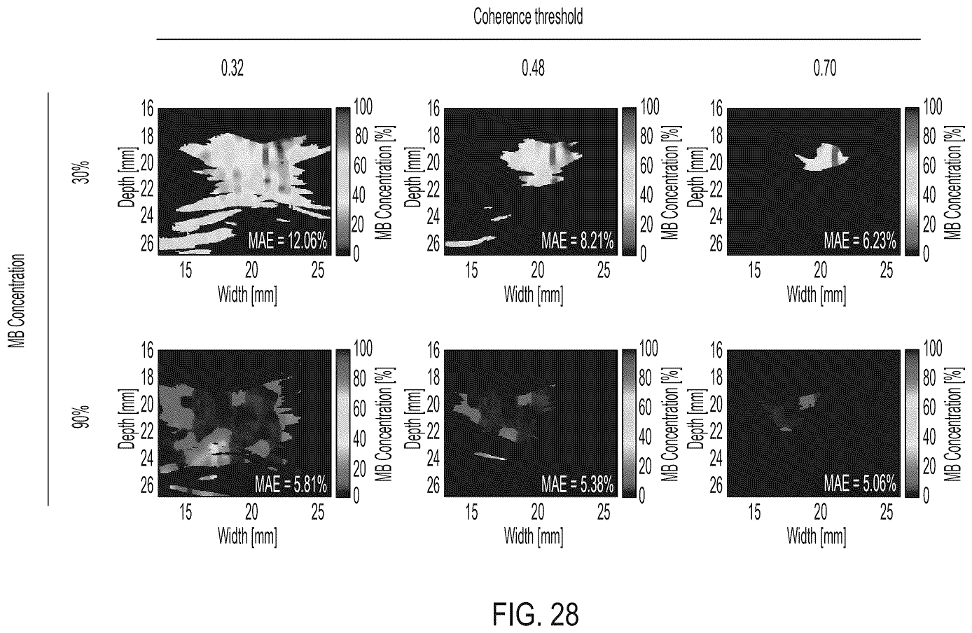

FIG. 28 shows examples of concentration maps with a mixture of 30% MB (top) and 90% MB (bottom) when using coherence thresholds of 0.32, 0.48, and 0.70 (from left to right, respectively). The measured MAE are reported in the lower right corner of each image. For the 30% MB mixture, when increasing the coherence threshold from 0.32 to 0.70, a MAE decrease from 12.06% to 6.23% (i.e., 5.83% decrease) is observed. The reduced error is attributed to absence of inaccurate estimates near the bottom of the 0.70 concentration map. In contrast, for the 90% MB mixture, when increasing the coherence threshold from 0.32 to 0.70, a MAE decrease of from 5.81% to 354 5.06% (i.e., 0.75% decrease) is observed, which can be attributed to a more uniform distribution of the mixture across the chamber. Overall, decreasing the mask size of the region of interest with more appropriate thresholding increases the performance of the dual-wavelength atlas method, as observed in FIG. 27 and FIG. 28.

### 11. DISCUSSION

A photoacoustic-based, dual-wavelength atlas method is disclosed that accurately estimates and generates concentration maps of a mixture of endogenous and exogenous chromophores (i.e., MB and Hb, respectively). The method builds on our previous dual-wavelength approach and measures the photoacoustic spectra obtained from two laser wavelength emissions as a linear combination of the chromophore spectra stored in an atlas. Linearity and monotonicity were confirmed with analyses of acoustic spectra and estimated concentrations as the ground-truth concentration increased, as shown in FIG. 23A and FIG. 23B and FIG. 24A and FIG. 24B, respectively.

FIG. 27 shows a mean absolute error of the estimated chromophore concentration obtained with the dual-wavelength atlas method as a function of M-weighted SLSC coherence thresholds.

FIG. 28 shows example concentration maps generated with different coherence threshold values.

Method optimization (see FIG. 23A) resulted in requiring only the first principal component for feature reduction, which agrees with previous optimization results of the dual-wavelength atlas method. However, the number principal component as well as the axial kernel size required for accurate estimation may increase with noise and diverse photoacoustic responses obtained from different factors, such as fiber tip geometries, vessel size, absorber size, ex vivo, and in vivo data.

There are three main factors that contribute to the variability of the estimated concentration maps shown in FIG. 26. First, the energy fluctuations provided by the laser system translated into unequal fluence among frames (e.g., the mean±one standard deviation of the laser energy at the fiber tip was 3.13±0.4 mJ for the human Hb experiment). Second, decreasing the coherence threshold chosen to segment meaningful photoacoustic signals when generating M-weighted SLSC masks decreased the estimation performance of the dual-wavelength atlas method, as shown in FIG. 27 and FIG. 28. Third, while precautions were taken to minimize differences in oxygen saturation among Hb samples, such variations were not negligible. Energy fluctuations among consecutive frames and difference in oxygen-saturation levels can be reduced when using fast-tuning lasers and fresh Hb from a single patient, respectively, in more realistic clinical scenarios.

When extending our approach to other ultrasound systems, implementation with a different transducer bandwidth would require an additional IQ calibration step to maximize chromophore concentration estimation performance. Typically, the IQ compression of radiofrequency signals is conducted using a modulation frequency equal to the central frequency of the ultrasound transducer. However, as observed in the stacked spectra shown in FIG. 22A and FIG. 22B, most of the frequency content resides within 1 to 4 MHz, while the corresponding transducer center frequency is 5.5 MHz. Therefore, the modulation frequency and bandwidth parameter of the IQ modulation step should be adjusted to segment meaningful spectra from the total bandwidth of the transducer and thus enhance the estimation performance of the present algorithm.

In vivo deployment of the dual-wavelength atlas method for mixture estimation requires two considerations. First, given that constructing a photoacoustic atlas is relatively fast (i.e., <10 minutes), extracting presurgical Hb samples from a single patient would be beneficial to minimize the estimation variability due to Hb samples from different patients at different draw times in the training set. Second, smaller vasculature are expected to yield different spectral responses because the frequency components are dependent on the volume of the chamber that is irradiated. For reference, the diameter of the PVCP chamber simulated the average size of the inferior vena cava (i.e., 0.46 to 2.26 cm in diameter). Therefore, vessel-specific parameter optimization is a possible future direction that can be explored with various chamber diameters.

### 12 CONCLUSION

The work described herein is the first to present an acoustic-based photoacoustic estimator that relies on training sets to estimate concentration levels from mixtures of photoacoustic-sensitive materials. The disclosed method comprises measuring the acoustic spectra obtained from two laser wavelength emissions as a linear combination of the chromophore spectra stored in an atlas. This linear combination assumption was confirmed with phantom experiments. In clinical practice, dual excitation wavelengths illuminating can be used in the region of interest with a fast-tuning laser source, providing real-time labeling of photoacoustic-sensitive regions with a parallelized version of the algorithm. The results from the presented experiments are promising for real-time monitoring of the concentration of contrast agents in the operating room.

In summary, an acoustic-based photoacoustic classifier that relies on training sets to identify photoacoustic-sensitive materials is disclosed. The disclosed method is robust against changes in fluence levels and showed comparable sensitivity, specificity, and accuracy performance to those obtained with conventional and enhanced spectral unmixing methods. In clinical practice, dual excitation wavelengths illuminating the region of interest with a fast-tuning laser source can be used, providing real-time labeling of photoacoustic-sensitive regions with a GPU-based parallelized algorithm version or deep neural network architectures. Results from the presented experiments are promising for the identification of biological or biocompatible markers (e.g., blood and contrast agents) during surgical interventions. By using the normalized photoacoustic response from two wavelength iterations, surgeons can localize structures of interest and surgical tools while avoiding other structures that are in close proximity to the targeted operating region.

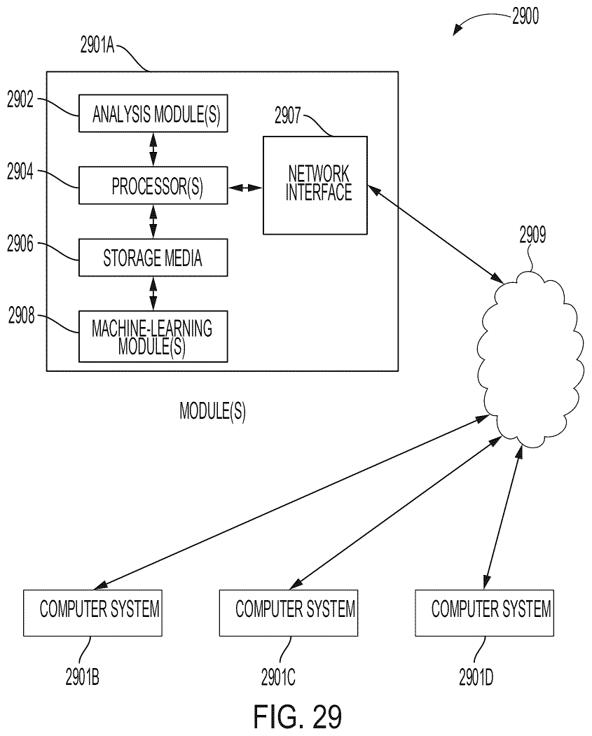

In some embodiments, any of the methods of the present disclosure may be executed by a computing system. FIG. 29 illustrates an example of such a computing system 2900, in accordance with some embodiments. The computing system 2900 may include a computer or computer system 2901A, which may be an individual computer system 2901A or an arrangement of distributed computer systems. The computer system 2901A includes one or more analysis module(s) 2902 configured to perform various tasks according to some embodiments, such as one or more methods disclosed herein. To perform these various tasks, the analysis module 2902 executes independently, or in coordination with, one or more processors 2904, which is (or are) connected to one or more storage media 1606. The processor(s) 2904 is (or are) also connected to a network interface 2907 to allow the computer system 2901A to communicate over a data network 2909 with one or more additional computer systems and/or computing systems, such as 2901B, 2901C, and/or 2901D (note that computer systems 2901B, 2901C and/or 2901D may or may not share the same architecture as computer system 2901A, and may be located in different physical locations, e.g., computer systems 2901A and 2901B may be located in a processing facility, while in communication with one or more computer systems such as 2901C and/or 2901D that are located in one or more data centers, and/or located in varying countries on different continents).

A processor can include a microprocessor, microcontroller, processor module or subsystem, programmable integrated circuit, programmable gate array, or another control or computing device.

The storage media 2906 can be implemented as one or more computer-readable or machine-readable storage media. The storage media 2906 can be connected to or coupled with a neuromodulation machine learning module(s) 2908. Note that while in the example embodiment of FIG. 29 storage media 2906 is depicted as within computer system 2901A, in some embodiments, storage media 2906 may be distributed within and/or across multiple internal and/or external enclosures of computing system 2901A and/or additional computing systems. Storage media 1606 may include one or more different forms of memory including semiconductor memory devices such as dynamic or static random access memories (DRAMs or SRAMs), erasable and programmable read-only memories (EPROMs), electrically erasable and programmable read-only memories (EEPROMs) and flash memories, magnetic disks such as fixed, floppy and removable disks, other magnetic media including tape, optical media such as compact disks (CDs) or digital video disks (DVDs), BLURAY® disks, or other types of optical storage, or other types of storage devices. Note that the instructions discussed above can be provided on one computer-readable or machine-readable storage medium, or alternatively, can be provided on multiple computer-readable or machine-readable storage media distributed in a large system having possibly plural nodes. Such computer-readable or machine-readable storage medium or media is (are) considered to be part of an article (or article of manufacture). An article or article of manufacture can refer to any manufactured single component or multiple components. The storage medium or media can be located either in the machine running the machine-readable instructions or located at a remote site from which machine-readable instructions can be downloaded over a network for execution.

It should be appreciated that computing system 2900 is only one example of a computing system, and that computing system 2900 may have more or fewer components than shown, may combine additional components not depicted in the example embodiment of FIG. 29, and/or computing system 2900 may have a different configuration or arrangement of the components depicted in FIG. 29. The various components shown in FIG. 29 may be implemented in hardware, software, or a combination of both hardware and software, including one or more signal processing and/or application specific integrated circuits.

Further, the steps in the processing methods described herein may be implemented by running one or more functional modules in an information processing apparatus such as general purpose processors or application specific chips, such as ASICs, FPGAs, PLDs, or other appropriate devices. These modules, combinations of these modules, and/or their combination with general hardware are all included within the scope of protection of the invention.

Analysis and/or material or part constraint data, models and/or other interpretation aids may be refined in an iterative fashion; this concept is applicable to embodiments of the present methods discussed herein. This can include use of feedback loops executed on an algorithmic basis, such as at a computing device (e.g., computing system 2900, FIG. 29), and/or through manual control by a user who may make determinations regarding whether a given step, action, template, model, or set of curves has become sufficiently accurate for the evaluation of the signal(s) under consideration.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. Moreover, the order in which the elements of the methods are illustrated and described may be re-arranged, and/or two or more elements may occur simultaneously. The embodiments were chosen and described in order to best explain the principles of the invention and its practical applications, to thereby enable others skilled in the art to best utilize the invention and various embodiments with various modifications as are suited to the particular use contemplated.

What is claimed is:

1. A method for identifying biological markers using acoustic frequency response, the method comprising:
obtaining acoustic information from a received signal from a target location;
classifying the acoustic information to provide classified acoustic information based on photoacoustic imaging response using a trained classifier; and
providing an indication of the classified acoustic information at the target location.

2. The method of claim 1, further comprising providing a single wavelength radiation emission or a dual-wavelength emission to the target location.

3. The method of claim 1, wherein the trained classifier is a neural network.

4. The method of claim 1, further comprising training a classifier to produce the trained classifier using a training data set of known photoacoustic-sensitive materials under a single wavelength emission conditions, a dual-wavelength emission conditions, or both.

5. The method of claim 4, wherein the training data set comprises biological material and contrast agents.

6. The method of claim 5, wherein the biological material comprises blood, lipid, collagen, or bone.

7. The method of claim 5, wherein the contrast agents comprise methylene blue or indocyanine green (ICG).

8. The method of claim 1, further comprising determining a concentration of each material within the target location based on the classified acoustic information.

9. The method of claim 1, further comprising alternative processing steps of the collected acoustic frequency information from two wavelength emission:
difference of the spectra obtained from each wavelength emission;
difference of the log compressed spectral obtained from each wavelength emission; and
cross correlation between the spectra obtained from each wavelength emission

10. The method of claim 1, further comprising of the use of a single light pulse containing two wavelengths for characterization of photoacoustic-sensitive materials through analysis of the frequency information of the acoustic response.

11. The method of claim 10, in addition to single or two wavelength emission, the use of a single pulse comprising two wavelengths for excitation is expected to offer the following benefits:
neglects the effect of motion artifacts in the imaging plane when switching between wavelength emissions, as only a single acquisition event is now configured;
reduces the acquisition time of photoacoustic data which benefits the feasibility of the algorithm for real-time implementations;
adds a third frequency response to the atlas which further enhances the differentiation between photoacoustic-sensitive materials; and
adds robustness in the characterization of the frequency content by the comparison of the new frequency response to the responses obtained from individual wavelengths.

12. A system for identifying biological markers using acoustic frequency response, the system comprising:
a hardware processor;
a non-transitory computer readable medium that stores instructions that when executed by the hardware processor perform a method comprising:
obtaining acoustic information from a received signal from a target location;
classifying the acoustic information to provide classified acoustic information based on photoacoustic imaging response using a trained classifier; and
providing an indication of the classified acoustic information at the target location.

13. The system of claim 12, wherein the method further comprises providing a single wavelength radiation emission or a dual-wavelength emission to the target location.

14. The system of claim 12, wherein the trained classifier is a neural network.

15. The system of claim 12, wherein the method further comprises training a classifier to produce the trained classifier using a training data set of known photoacoustic-sensitive materials under a single wavelength emission conditions, a dual-wavelength emission conditions, or both.

16. The system of claim 15, wherein the training data set comprises biological material and contrast agents.

17. The system of claim 16, wherein the biological material comprises blood, lipid, collagen, or bone.

18. The system of claim 16, wherein the contrast agents comprise methylene blue and indocyanine green (ICG).

19. The system of claim 12, wherein the method further comprises determining a concentration of each material within the target location based on the classified acoustic information.
